(19) United States
(12) Patent Application Publication
(10) Pub. No.: US 2024/0086771 A1
(43) Pub. Date: Mar. 14, 2024

(54) MACHINE LEARNING TO GENERATE SERVICE RECOMMENDATIONS

(71) Applicant: MatrixCare, Inc., Bloomington, MN (US)

(72) Inventors: Vivek KUMAR, Eden Prairie, MN (US); Samsudhin H., Chennai (IN); Nivedita SINGH, Ahmedabad (IN)

(21) Appl. No.: 18/465,473

(22) Filed: Sep. 12, 2023

Related U.S. Application Data
(60) Provisional application No. 63/375,287, filed on Sep. 12, 2022.

(51) Int. Cl. G06N 20/00

(52) U.S. Cl. CPC G06N 20/00

(57) ABSTRACT

Techniques for improved machine learning are provided. Resident data describing a resident is accessed, and a residential service plan is generated for the resident, comprising extracting a set of features from the resident data and generating a set of predicted fitness scores for a set of services by processing the set of features using a machine learning model trained based on one or more collaborative filtering techniques. The residential service plan is implemented for the resident based at least in part on the set of predicted fitness scores.

### CROSS-REFERENCE TO RELATED APPLICATIONS

This application claims priority to U.S. Provisional Patent Application No. 63/375,287, filed Sep. 12, 2022, the entire content of which is incorporated herein by reference in its entirety.

### INTRODUCTION

Embodiments of the present disclosure relate to machine learning. More specifically, embodiments of the present disclosure relate to using machine learning to generate and evaluate residential service plans in healthcare settings.

In many healthcare settings, such as in residential care facilities (e.g., nursing homes or senior care facilities), a wide variety of user, patient, or resident characteristics are assessed and monitored in an effort to reduce or prevent a resident's condition from worsening. Additionally, various sets of services are often devised by clinicians or other healthcare providers in an effort to ameliorate any issues or concerns for the user. For example, service plans may be used to ensure that the user receives any needed assistance (e.g., help with eating). However, such service plans have a multitude of varying alternatives and options, and are tremendously complex to design. Without appropriate service planning, these problems can lead to clinically significant negative outcomes and complications.

Conventionally, healthcare providers (e.g., doctors, nurses, caregivers, and the like) strive to provide adequate healthcare service planning using manual assessments (e.g., relying on subjective experience) and/or explicit indications, from the residents or their relatives, about the services needed. However, such conventional approaches are entirely subjective (relying on the expertise of individual caregiver to recognize and care for possible concerns, and/or on the biased assessment of the resident or family members), and frequently fail to identify the most optimal service plans for a variety of residents. Further, given the vast complexity involved in these plans, it is simply impossible for healthcare providers to evaluate all relevant data and alternatives in order to select optimal plans.

Improved systems and techniques to automatically generate and/or evaluate service plans are needed.

### SUMMARY

According to one embodiment presented in this disclosure, a method is provided. The method includes: accessing resident data describing a set of services received by a resident; generating a set of fitness scores for the set of services, wherein each respective fitness score from the set of fitness scores indicates a respective suitability of a respective service for the resident; training a machine learning model to generate residential service plans based at least in part on the set of fitness scores using one or more collaborative filtering techniques; and deploying the trained machine learning model.

According to one embodiment presented in this disclosure, a method is provided. The method includes: accessing resident data describing a resident; generating a residential service plan for the resident, comprising: extracting a set of features from the resident data; and generating a set of predicted fitness scores for a set of services by processing the set of features using a machine learning model trained based on one or more collaborative filtering techniques; and implementing the residential service plan for the resident based at least in part on the set of predicted fitness scores.

According to one embodiment presented in this disclosure, a system is provided. The method includes one or more computer processors, and one or more memories containing a program which when executed by the one or more computer processors performs an operation. The operation includes: accessing resident data describing a resident; generating a residential service plan for the resident, comprising: extracting a set of features from the resident data; and generating a set of predicted fitness scores for a set of services by processing the set of features using a machine learning model trained based on one or more collaborative filtering techniques; and implementing the residential service plan for the resident based at least in part on the set of predicted fitness scores, comprising, for each respective service of the set of services: selecting a manner of presentation based on a corresponding predicted fitness score from the set of predicted fitness scores; and generating a visual depiction, on a graphical user interface (GUI), of suitability of the respective service for the resident based on the selected manner of presentation.

Other embodiments presented in this disclosure provide processing systems configured to perform the aforementioned methods as well as those described herein; non-transitory, computer-readable media comprising instructions that, when executed by a processor of a processing system, cause the processing system to perform the aforementioned methods as well as those described herein; a computer program product embodied on a computer-readable storage medium comprising code for performing the aforementioned methods as well as those further described herein; and a processing system comprising means for performing the aforementioned methods as well as those further described herein.

The following description and the related drawings set forth in detail certain illustrative features of one or more embodiments.

### DESCRIPTION OF THE DRAWINGS

The appended figures depict certain aspects of the one or more embodiments and are therefore not to be considered limiting of the scope of this disclosure.

FIG. 1 depicts an example workflow for training machine learning models based on historical data.

FIG. 2 depicts an example workflow for generating training data to drive improved service planning.

FIG. 3 depicts an example workflow for generating service plans using trained models.

FIG. 4 depicts an example workflow for generating mappings and training models to suggest resident services.

FIG. 5 depicts an example workflow for generating suggested service plans.

FIG. 6 depicts a flow diagram depicting an example method for training machine learning models to generate service plans.

FIG. 7 depicts a flow diagram depicting an example method for extracting features to drive model training for service plan generation.

FIG. 8 is a flow diagram depicting an example method for scoring historical services to generate training data for machine learning models.

FIG. 9 is a flow diagram depicting an example method for generating mappings for improved machine learning to generate service plans.

FIG. 10 is a flow diagram depicting an example method for generating service plans using machine learning.

FIG. 11 is a flow diagram depicting an example method for scoring and ranking services using trained models.

FIG. 12 is a flow diagram depicting an example method for updating graphical user interfaces (GUIs) based on machine learning model output for service plan generation.

FIG. 13 is a flow diagram depicting an example method for generating facility-wide service plan data using machine learning.

FIG. 14 is a flow diagram depicting an example method for training machine learning models to generate residential service plans.

FIG. 15 is a flow diagram depicting an example method for generating residential service plans using machine learning models.

FIG. 16 depicts an example computing device configured to perform various aspects of the present disclosure.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Aspects of the present disclosure provide apparatuses, methods, processing systems, and computer-readable mediums for improved machine learning to generate and evaluate residential service plans, such as for residents in long-term residential care facilities.

In some embodiments, a machine learning model (also referred to in some aspects as a service model) can be trained and used as a tool for clinicians (e.g., nurses, caregivers, doctors, and the like) to assist in generating and evaluating residential service plans for residents (also referred to in some aspects as users or patients), thereby improving care, and preventing potentially significant negative outcomes. In some embodiments, by using objective models generated using historical data, the system is able to enable efficient allocation of resources and provide specific interventions to help mitigate, prevent, or reduce the effect of a myriad of problems and disorders for the residents.

In conventional settings, care providers must rely on subjective assessments and plans (e.g., manually selecting service plans) and on the biased and subjective suggestions of the resident and/or resident families to identify suitable service plans. In addition to this inherently subjective and inaccurate approach, many conventional systems are largely static and have difficultly responding to dynamic situations (e.g., changing resident conditions) which are common in practice. Moreover, the vast number and variety of alternative services that can be used (and the various degrees or types of services within each alternative service), as well as the significant amounts of data available for each resident, render accurate analysis and selection of proper plans impossible to adequately perform manually or mentally. Aspects of the present disclosure can not only reduce or prevent this subjective review, but can further prevent wasted time and computational expense spent reviewing vast amounts of irrelevant data and sub-optimal plans. Further, aspects of the present disclosure enable more accurate evaluations, more efficient use of computational and other resources (e.g., reducing or eliminating frequent requests for information, and efficiently evaluating vast stores of data), and overall improved outcomes for residents (e.g., through reduced harm to the resident).

Embodiments of the present disclosure can generally enable proactive and quality care for residents, as well as dynamic and targeted interventions, that help to prevent or reduce adverse events due to a variety of issues and conditions. This autonomous and continuous updating based on changing conditions with respect to individual residents enables a wide variety of improved results, including not only improved outcomes for the residents themselves (e.g., reduced negative outcomes, early identification of optimal plans, targeted interventions, and the like) but also improved computational efficiency and accuracy of the evaluation and solution process.

In some embodiments, a variety of historical resident data can be collected and evaluated to train one or more machine learning models. During such training, the machine learning model(s) can learn a set of features (e.g., resident attributes) and/or a set of weights for such features. These features and weights can then be used to automatically and efficiently process new resident data in order to generate and evaluate improved service plans. In some aspects, a modified collaborative filtering approach is introduced to improve the accuracy and reliability of the model, while simultaneously improving the computational efficiency of training and using the model, as compared to conventional approaches.

In at least one embodiment, the model can be trained based on historical service data, including information such as the amount of time spent providing a given service to a resident, natural language notes relating to the provisioning of services, completion statuses for prior services, and the like to learn which service(s) are most suitable for a given resident based on their individual characteristics (e.g., their demographics, conditions, medications, and the like). These models can then be used to drive service selection for new residents (e.g., during admission) as well as during the resident's stay in the facility (e.g., periodically, or when their status changes).

### Example Workflow for Training Machine Learning Models Using Historical Data

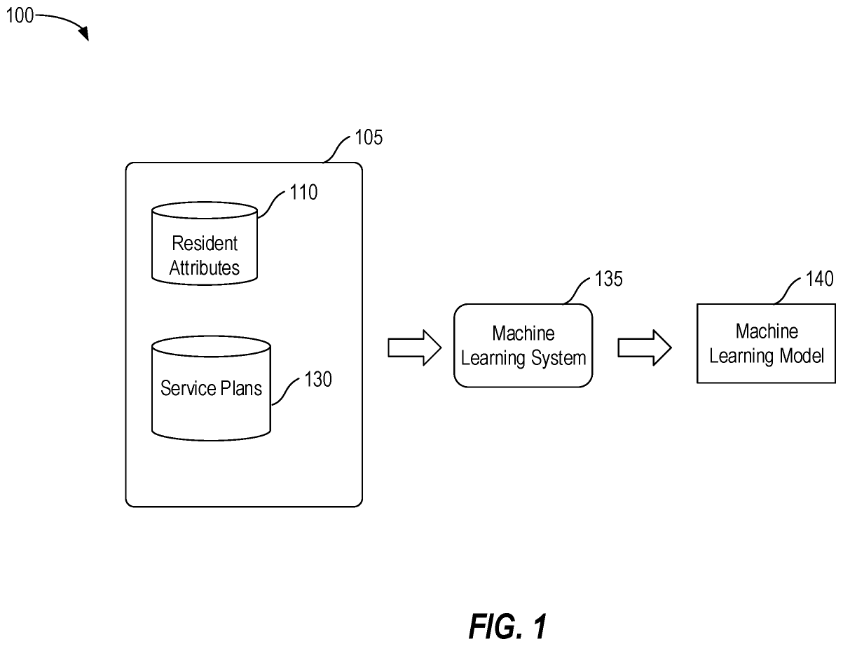

FIG. 1 depicts an example workflow 100 for training machine learning models based on historical data.

In the illustrated workflow 100, a set of historical data 105 is evaluated by a machine learning system 135 to generate one or more machine learning models 140. In embodiments, the machine learning system 135 may be implemented using hardware, software, or a combination of hardware and software. The historical data 105 generally includes data or information associated with one or more residents in one or more residential care facilities from one or more prior points in time. That is, the historical data 105 may include, for one or more residents, a set of one or more snapshots of the resident's characteristics or attributes at one or more points in time. Generally, the historical data 105 includes data relating to services that were previously provided to one or more residents in one or more facilities.

As illustrated the historical data 105 includes information relating to resident attributes 110 and service plans 130. The resident attributes 110 generally include information relating to the attributes or characteristics of residents in one or more residential-care facilities, such as their demographics (e.g., sex or gender, race, marital status, age, and the like), any diagnoses or conditions they have, any medications they use, any allergies they have, and the like. In some aspects, as discussed below in more detail, the resident attributes 110 are use as input to the machine learning model in order to generate suggested service plans (e.g., a set of services, or indications as to which service(s) are likely needed).

In the illustrated example, the historical data 105 further includes information relating to one or more service plans 130 used by residents in one or more residential facilities. In an aspect, each service plan 130 is associated with a corresponding resident (e.g., a resident reflected in the resident attributes 110) and/or time, such that the relevant resident attributes 110 for any given service plan 130 can be readily determined (e.g., the attributes of the corresponding resident at the corresponding time). The service plans 130 generally include information such as the specific service(s) used, the completion status(es) of each service used at one or more points in time, the service minutes or durations (e.g., the amount of time that a caregiver spent to provide each service at one or more points in time), any recorded exceptions or flags (e.g., added by a caregiver after providing, or attempting to provide, each service), natural language comments or notes about the service (e.g., written by a caregiver), and the like.

In some aspects, as discussed in more detail below, the service plans 130 can be evaluated to generate, for each service reflected in a given plan, a fitness score indicating the suitability or necessity for the given service at the given time and for the given resident. For example, the system may score each prior-provided service (e.g., between zero and one, or between zero and ten), where lower scores generally indicate low fitness or suitability (e.g., indicating that the service was not needed or desired by the resident at the time) and higher scores indicate higher fitness or suitability (e.g., indicating that the service was needed by the resident at the time). In this way, the fitness scores can be used as a target variable to train one or more models, as discussed in more detail below.

As some (non-limiting) examples of alternative services that may be included in the historical data 105 and/or suggested by the machine learning model 140, the service plans 130 may include information relating to services such as activities of daily living (e.g., dressing and grooming, bathing, bathroom assistance, eating, and the like), cognitive or psychosocial assistance (e.g., orientation issues, wandering behavior, communication problems, socialization, mobility, and the like), housekeeping assistance (e.g., general housekeeping, laundry, and the like), nutrition or dining services (e.g., diet assistance, meal preparation, and the like), health-related services (e.g., due to chronic illness, medication assistance, skin care assistance, and the like), emergency services (e.g., emergency evacuation assistance, use or installation of an emergency pull cord or other alert device, and the like), as well as any other resident services (such as plant care, pet care, finance management, shopping assistance, and the like).

In some aspects of the present disclosure, the various components of the historical data 105 are described with reference to a single resident at a single time for conceptual clarity (e.g., resident attributes 110 of a single resident having a single service plan 130 at a single time) for conceptual clarity. However, it is to be understood that the historical data 105 can generally include such data for any number of residents and plans.

In the illustrated example, the historical data 105 is accessed for processing by a machine learning system 135. Although depicted as a discrete system for conceptual clarity, in aspects, the functionality of the machine learning system 135 may be implemented using hardware, software, or a combination of hardware and software, and may be implemented as a standalone system or as a component of a broader system.

Although the illustrated workflow 100 depicts the machine learning system 135 accessing the historical data 105 from an external repository, in some aspects, some or all of the historical data 105 may reside or be maintained locally by the machine learning system 135. Additionally, though the illustrated example suggests a single repository of historical data 105 for conceptual clarity, in aspects, the various data included in the historical data 105 may reside in any number of repositories or data sources, depending on the particular implementation. As used herein, “accessing” data (such as the historical data 105) can generally include receiving, requesting, retrieving, or otherwise gaining access to the data.

In some embodiments, as discussed in more detail below, the machine learning system 135 can preprocess and use the historical data 105 to generate or train the machine learning model 140. Although a single machine learning model 140 is depicted for conceptual clarity, in some aspects, the machine learning system 135 may generate multiple such models. For example, the machine learning system 135 may generate region-specific models (e.g., a respective machine learning model 140 for each region, country, or other locale), where each model is trained based on historical data 105 associated with the specific region. This can improve the model performance in some aspects.

In embodiments, the architecture of the machine learning model 140 may vary depending on the particular implementation. For example, in some embodiments, the machine learning model 140 may be a neural network model, a random forest model, and the like. In some embodiments, the machine learning system 135 uses a modified collaborative filtering approach to train the machine learning model 140, as discussed in more detail below. For example, while conventional approaches to collaborative filtering generally involve evaluating either user-to-user similarity or item-to-item similarity, some aspects of the present disclosure provide collaborative filtering techniques that group and/or combine similarities in order to suggest appropriate or optimal residential services, as discussed in more detail below.

Generally, to train the machine learning model 140, one or more resident attribute(s) 110 are used as input data, while the generated fitness scores (created based on the service plans 130) are used as the target variable or output. In this way, the machine learning model 140 learns to generate or suggest proposed or alternative services, for a given resident, based on their attributes. In some aspects, the model generates a score and/or rank for each alternative service, enabling users (e.g., clinicians or healthcare providers) to identify the optimal set of services for the resident at the specific time based on their specific characteristics.

In some embodiments, the machine learning system 135 outputs the machine learning model 140 to one or more other systems for use. That is, the machine learning system 135 may distribute the machine learning model 140 to one or more downstream systems, where each downstream system is used to generate service plans. For example, the machine learning system 135 may deploy the machine learning model 140 to one or more servers associated with specific residential care facilities, and these servers may use the model to evaluate potential service plans for residents. In at least one embodiment, the machine learning system 135 can itself use the machine learning model to generate service plans.

### Example Workflow for Generating Training Data to Drive Improved Service Planning

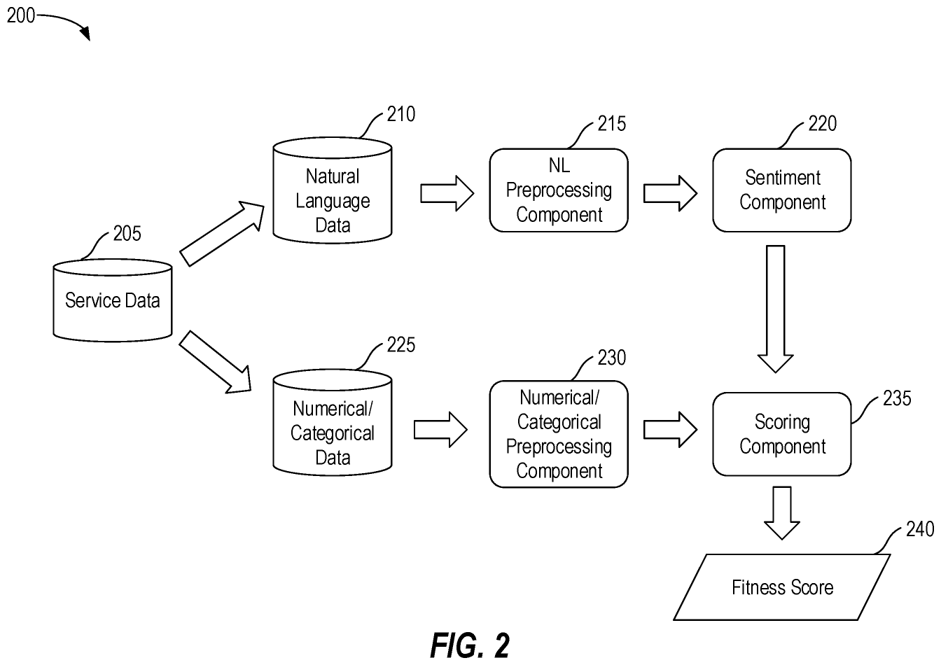

FIG. 2 depicts an example workflow 200 for generating training data to drive improved service planning. In some embodiments, the workflow 200 is used to generate the training data used to train machine learning models (e.g., using the workflow 100 of FIG. 1). In some embodiments, the workflow 200 is performed by a machine learning system, such as the machine learning system 135 of FIG. 1.

In the illustrated example, service data 205 is received or accessed for processing in order to generate a fitness score 240 for each service reflected in the service data 205. As discussed above, the fitness score 240 generally indicates a suitability or appropriateness of providing a given service to a given user at a given time. For example, a high fitness score 240 may indicate that the service is highly appropriate or suitable (e.g., the resident needed the service) while a low fitness score 240 may indicate a low suitability (e.g., the resident was able to complete the task without assistance, did not need to complete the task at all (e.g., if the resident does not take any medications, but the service was for medication assistance), or the service was otherwise inappropriate or not needed.

In some embodiments, the service data 205 may correspond to the service plans 130 of FIG. 1. In some aspects, the service data 205 includes information for prior or historical services that have been used, provided, or completed in a residential care facility. For example, the service data 205 may include a set of records, each record including information relating to one or more times when one or more services were provided to one or more residents. For example, the service data 205 may include, for each prior-rendered service, information such as the completion status(es) at one or more points in time, the service minutes or durations (e.g., the amount of time that a caregiver spent to provide each service at one or more points in time), any recorded exceptions or flags (e.g., added by a caregiver after providing, or attempting to provide, each service), natural language comments or notes about the service (e.g., written by a caregiver), and the like.

In the illustrated workflow 200, the service data 205 can be delineated into natural language data 210 and numerical or categorical data 225. Although the illustrated examples depicts an actual physical or discrete division of the service data 205 (e.g., transferring some data to the natural language data 210 and other data to the numerical/categorical data 225) for conceptual clarity, in aspects, the machine learning system may access or operate on the service data 205 without such transfer. For example, the natural language (NL) preprocessing component 215 may extract the relevant natural language data 210 directly from the service data 205 for processing.

In an embodiment, the natural language data 210 can include natural language text, such as written by a caregiver in a note or comment field to indicate or describe how providing the service proceeded. For example, the caregiver may record a note such as “Refused to have meals today. Said he was too tired,” “Transfer from bed to wheelchair and then escorted to the dining room for breakfast,” “resident refused meds all morning then accepted in the afternoon,” “made the bed, did the dishes, emptied the trash, did one load of laundry,” and the like. Generally, the natural language data 210 can include any natural language description of the process of providing one or more services (whether or not the service is actually provided or completed) to a resident, or otherwise assessing or describing the state of the resident when the service was provided (or attempted to be provided).

In some embodiments, the natural language data 210 can generally include textual data. For example, the caregiver may use a keyboard or other input device to type the comments or notes. Similarly, in some embodiments, the textual data may be obtained by processing recorded audio (e.g., speech captured by a microphone) using one or more speech-to-text algorithms, and/or may be obtained by processing handwritten notes using one or more optical character recognition (OCR) techniques.

As illustrated, the natural language data 210 is processed by a NL preprocessing component 215. Though depicted as a discrete component for conceptual clarity, in embodiments, the operations of the NL preprocessing component 215 may be combined or distributed across any number and variety of components, and may be implemented using hardware, software, or a combination of hardware and software. The NL preprocessing component 215 can generally provide a variety of preprocessing operations, and may be used during training of machine learning models (e.g., to generate the training input). The NL preprocessing component 215 may generally be used to transform or preprocess any natural language input, prior to it being used as input to the machine learning model(s).

In some embodiments, the operations of the NL preprocessing component 215 are referred to as preprocessing to indicate that they are used to transform, refine, manage, or otherwise modify the natural language data 210 to improve its suitability for use with machine learning systems (or other downstream processing). For example, in some embodiments, preprocessing the data in the natural language data 210 may improve the training process by making the data more compatible with natural language processing, and ultimately for consumption by the model during training. Preprocessing can generally include a variety operations, which may include a series of operations performed sequentially and/or in parallel.

The NL preprocessing component 215 may generally implement any suitable preprocessing operations, including (but not limited to) text extraction, normalization, noise removal, redundancy removal, lemmatization, tokenization, root generation, vectorization, and the like.

Generally, text extraction may correspond to extracting natural language text from an unstructured portion of the natural language data 210. For example, if the natural language data 210 includes a set of notes (e.g., notes written by a resident indicating reason(s) for one or more services, or written by a caregiver providing the service), the text extraction can include identifying and extracting these notes for evaluation.

Generally, normalization can include a wide variety of text normalization processes, such as converting all characters in the extracted text to lowercase, converting accented or foreign language characters to ASCII characters, expanding contractions, converting words to numeric form where applicable, converting dates to a standard date format, and the like.

Generally, noise removal can include identification and removal of portions of the extracted text that do not carry meaningful or probative value. That is, noise removal may include removing characters, portions, or elements of the text that are not useful or meaningful in the ultimate computing task (e.g., evaluating the sentiment or complexity of the note and/or of the service), and/or that are not useful to human readers. For example, the noise removal may include removing extra white or blank spaces, tabs, or lines, removing tags such as HTML tags, and the like.

Generally, redundancy removal may correspond to identifying and eliminating or removing text corresponding to redundant elements (e.g., duplicate words), and/or the reduction of a sentence or phrase to a portion thereof that is most suitable for machine learning training or application. For example, the redundancy removal may include eliminating verbs (which may be unhelpful in the machine learning task), conjunctions, or other extraneous words that do not aid the machine learning task.

Generally, lemmatization can include stemming and/or lemmatization of one or more words in the extracted text. This may include converting words from their inflectional or other form to a base form. For example, lemmatization may include replacing “holding,” “holds,” and “held” with the base form “hold.”

Generally, tokenization 435 includes transforming or splitting elements in the extracted text (e.g., strings of characters) into smaller elements, also referred to as “tokens.” For example, the tokenization 435 may include tokenizing a paragraph into a set of sentences, tokenizing a sentence into a set of words, transforming a word into a set of characters, and the like. In some embodiments, tokenization can additionally or alternatively refer to the replacement of sensitive data with placeholder values for downstream processing. For example, text such as the personal address of the resident may be replaced or masked with a placeholder (referred to as a “token” in some aspects), allowing the remaining text to be evaluated without exposing this private information.

Generally, root generation can include reducing a portion of the extracted text (e.g., a phrase or sentence) to its most relevant n-gram (e.g., a bigram) or root for downstream machine learning training and/or application.

Generally, vectorization may include converting the text into one or more objects that can be represented numerically (e.g., into a vector or tensor form). For example, the vectorization may use one-hot encodings (e.g., where each element in the vector indicates the presence or absence of a given word, phrase, sentiment, or other concept, based on the value of the element). In some embodiments, the vectorization can correspond to any word or sentence embedding vectors (e.g., generated using all or a portion of a trained machine learning model, such as the initial layer(s) of a feature extraction model). This resulting object can then be processed by downstream natural language processing algorithms or machine learning models to improve the ability of the system to evaluate the text (e.g., to drive more accurate sentiment or complexity scores).

In the illustrated example, the resulting preprocessed NL data (output by the NL preprocessing component 215) can be provided to a sentiment component 220 for analysis. Though depicted as a discrete component for conceptual clarity, in embodiments, the operations of the sentiment component 220 may be combined or distributed across any number and variety of components, and may be implemented using hardware, software, or a combination of hardware and software. The sentiment component 220 can be used to evaluate natural language text (which may be preprocessed as discussed above) to generate a score representing the complexity or sentiment reflected in the text.

In some embodiments, the sentiment component 220 uses a trained machine learning model to generate a complexity score based on the text, where the complexity score indicates how complex or difficult providing the service was (e.g., from zero to one, from zero to ten, and the like). These sentiment/complexity model(s) may be trained using labeled exemplars (e.g., where each training sentence or phrase has a corresponding score indicating how complex the task was, based on the comments). For example, a note indicating that the service was provided without difficulty (e.g., “resident took medications immediately, after a glass of water was provided”) may be scored relatively lower, by the sentiment component 220, than a note indicating that the caregiver was met with more difficulty when providing the service (e.g., “resident refused medications throughout the morning, and finally accepted medication when resident's daughter visited this afternoon and presented them”). As illustrated, this complexity score is then provided to a scoring component 235 for processing, as discussed in more detail below.

In the illustrated example, the numerical/categorical data 225 can generally any data that is not natural language text, and can include structured data, unstructured data, numerical data (e.g., data with integer values, continuous values, ranges of values, and the like), categorical data (e.g., where the data has a specific class or category), and the like. In at least one embodiment, the numerical/categorical data 225 can include information such as the service completion response or flag (e.g., indicating whether the service was completed, not completed, or completed with exceptions/flags), a service duration (e.g., the number of minutes spent providing the service), any exception responses or flags, if included (e.g., indicating that the resident is sick, indicating an other unspecified exception, indicating that the resident refused the service, indicating that the resident was absent or not present when the caregiver arrived, indicating that the resident required more assistance than the caregiver was able to provide, indicating that one or more assistive devices, such as bed lift mechanisms, were needed to provide the service, and the like).

In the depicted workflow 200, the numerical/categorical data 225 can optionally be provided to a numerical/categorical preprocessing component 230 for preprocessing. Though depicted as a discrete component for conceptual clarity, in embodiments, the operations of the numerical/categorical preprocessing component 230 may be combined or distributed across any number and variety of components, and may be implemented using hardware, software, or a combination of hardware and software. The numerical/categorical preprocessing component 230 can generally provide a variety of preprocessing operations for the data, such as converting categorical data to a numerical score (e.g., assigning a score to each exception response flag based on a defined mapping, assigning a score to each completion status category based on a defined mapping, and the like), converting or transforming a numerical score (e.g., dividing the number of minutes spent providing the service by a defined value, such as five), and the like. In some aspects, some or all of such transformations may alternatively be performed by the scoring component 235. That is, the numerical/categorical data 225 may be provided directly to the scoring component 235.

In the illustrated example, the scoring component 235 receives a sentiment or complexity score (from the sentiment component 220) and one or more categorical or numerical values (from the numerical/categorical data 225 and/or from the numerical/categorical preprocessing component 230). Though depicted as a discrete component for conceptual clarity, in embodiments, the operations of the scoring component 235 may be combined or distributed across any number and variety of components, and may be implemented using hardware, software, or a combination of hardware and software.

The scoring component 235 can generally evaluate the input data to generate an overall fitness score 240 for each specific service that was provided, as indicated in the service data 205. In some aspects, the scoring component 235 uses one or more trained models to generate an output score based on the inputs. In other embodiments, the scoring component 235 may use one or more algorithms or aggregation techniques to generate a score.

For example, in one aspect, the scoring component 235 can determine and/or generate a numerical score or value (also referred to as a subscore in some aspects) for each feature used to generate the fitness score 240, multiply this subscore using a feature-specific weight (which may be learned or manually-defined), and aggregate the scores (e.g., using summation) to generate an overall fitness score 240 for the service. As an example, the completion status may be assigned a subscore based on a defined mapping (e.g., where a “completed” status maps to one score, and “not completed” maps to a second, relatively higher, score), the exception flag(s) may be assigned a subscore based on a mapping (e.g., where exception flags indicative of additional complexity, effort, or need for the service are associated with higher subscores), and the like. In at least one aspect, the amount of time needed to complete the service, as well as the complexity or sentiment score, are directly related to the fitness score 240 (e.g., where higher durations/scores result in higher fitness scores 240).

As discussed above, the fitness score 240 can then be used as a target variable in the training data used to train, refine, or otherwise generate service models. For example, in one embodiment, the resident's attributes may be used as input to generate a numerical value indicating how suitable or appropriate a given service, and this value can be compared against the fitness score 240 to generate a loss that is used to refine the model.

In some embodiments, as discussed above, the fitness scores 240 are used in a collaborative filtering model approach, where the fitness scores 240 act as the ground-truth or label for the data (e.g., indicating how appropriate or optimal a given service is for the given resident), as discussed in more detail below.

### Example Workflow for Generating Service Plans Using Trained Models

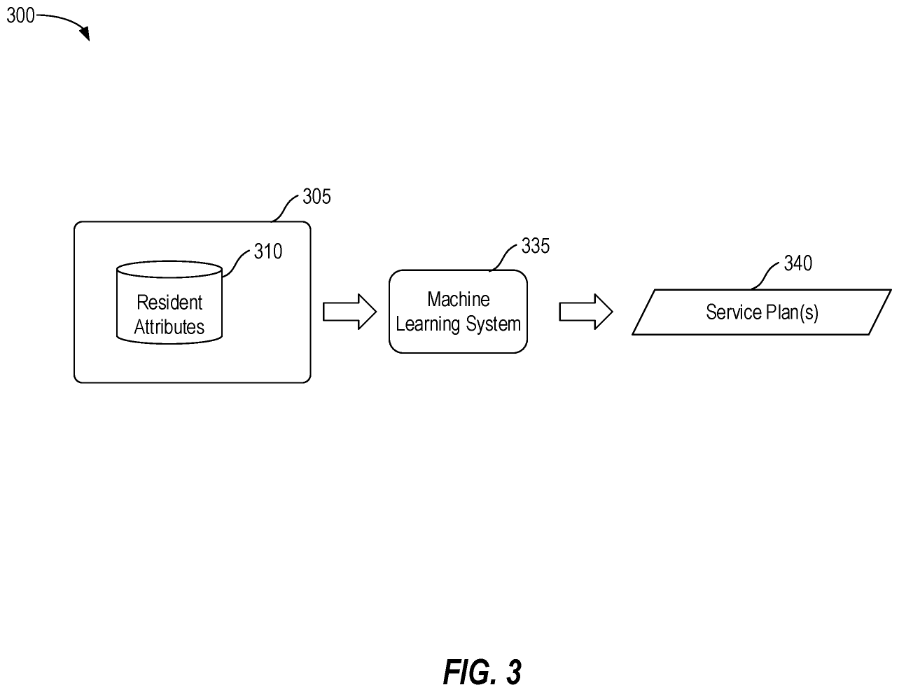

FIG. 3 depicts an example workflow for generating 300 service plans using trained models. In some embodiments, the workflow 300 is performed using trained models, such as the machine learning models 140 of FIG. 1.

In the illustrated workflow 300, a set of resident data 305 is evaluated by a machine learning system 335 using one or more machine learning models (e.g., machine learning model 140 of FIG. 1) to generate and/or evaluate one or more service plan(s) 340. In embodiments, the machine learning system 335 may be implemented using hardware, software, or a combination of hardware and software. In some embodiments, the machine learning system 335 corresponds to the machine learning system 135 of FIG. 1. In other aspects, as discussed above, the machine learning system 335 may differ from the system that trained the model and/or generated the training data.

The resident data 305 generally includes data or information associated with one or more residents, as discussed above. That is, the resident data 305 may include, for one or more residents in one or more residential facilities, a set of one or more snapshots of their characteristics or attributes at one or more points in time. In the illustrated example, the resident data 305 includes, for each resident reflected in the data, a set of one or more resident attributes 310.

As discussed above, the resident attributes 310 generally include information relating to a set of one or more specified features, attributes, or characteristics describing the resident(s), such as their demographics, diagnoses, medication(s), allergies, and the like. In some embodiments, the resident data 305 corresponds to new residents (e.g., new users that are entering or being admitted to the facility). In some embodiments, the resident data 305 can additionally or alternatively include data for current residents (e.g., users that already reside in a facility).

In the illustrated example, the machine learning system 335 can access the resident attributes 310 of each respective resident in order to generate a corresponding service plan 340. In some aspects, as discussed above, the machine learning system 335 uses a collaborative filtering-based model, trained based on prior resident service data, to identify and suggest individual services within the service plan 340.

In some embodiments, the machine learning system 335 generates, for each potential service of a set of potential services that may be offered to the resident, a respective score or ranking (e.g., a fitness score) indicating whether the service is likely to be appropriate or suitable for the resident (based on the resident's attributes). For example, in some aspects, the machine learning system 335 evaluates all possible services (e.g., a set of all services offered by the facility). In some embodiments, the machine learning system 335 can first filter or sort the potential services (e.g., using collaborative filtering) to find a subset of services that may be useful for the specific resident, based on the resident attributes 310. The machine learning system 335 may then score or rank these services as discussed in more detail below (e.g., based on the generated fitness scores from prior training data).

In some embodiments, the machine learning system 335 can identify or suggest any services having a score that meets or exceeds a defined threshold (or otherwise satisfies defined criteria). For example, for any services with a score that meets the criteria, the machine learning system 335 may add this service to the service plan 340. In some embodiments, the machine learning system 335 may output some or all of the potential services, along with the predicted fitness scores, to a user (e.g., a caregiver), allowing the user to select which service(s) should be included in the service plan 340.

In some embodiments, the machine learning system 335 can automatically implement the service plan(s) 340. For example, the machine learning system 335 may automatically create work orders, engage with contractors, generate instructions to caregivers, generate invoices, and the like, in order to generally perform any operations used to instantiate the service plan 340 or begin providing any of the services. In some embodiments, the machine learning system 335 can additionally or alternatively output the service plan 340 to a user, allowing them to review and/or approve it.

In some embodiments, the machine learning system 335 can generate a new service plan 340 for each resident periodically (e.g., weekly). In some embodiments, the machine learning system 335 generates a new service plan 340 whenever new data becomes available (or when the resident data 305 changes). For example, when a new diagnosis is reported for a resident, the machine learning system 335 may automatically detect the change, extract the updated resident attribute(s) 310, and generate an updated service plan 340 that is specifically-tailored to the individual resident at the specific time. This targeted prophylactic treatment can significantly improve resident conditions and reduce harm.

Advantageously, the automatically generated service plans 340 can significantly improve the outcomes of the residents, helping to identify optimal plans, thereby preventing further deterioration and significantly reducing harm. Additionally, the autonomous nature of the machine learning system 335 enables improved computational efficiency and accuracy, as the service plans 340 can be generated objectively (as opposed to the subjective judgment of clinicians or other users), as well as quickly and with minimal computational expense. That is, as the plans can be automatically updated whenever new data is available, users need not manually retrieve and review the relevant data (which incurs wasted computational expense, as well as wasted time for the user).

Further, in some embodiments, the machine learning system 335 can regenerate service plans 340 during specified times (e.g., off-peak hours, such as overnight) to provide improved load balancing on the underlying computational systems. For example, rather than requiring service providers to retrieve and review resident data to determine if anything occurred or changed (which may require a new service plan), the machine learning system 335 can automatically identify such changes, and use the machine learning model(s) to regenerate service plans 340. This can transfer the computational burden, which may include both processing power of the storage repositories and access terminals, as well as bandwidth over one or more networks, to off-peak times, thereby reducing congestion on the system during ordinary (e.g., daytime) use and taking advantage of extra resources that are available during the non-peak (e.g., overnight) hours.

In these ways, embodiments of the present disclosure can significantly improve resident outcomes while simultaneously improving the operations of the computers and/or networks themselves (at least through improved and more accurate scores, as well as better load balancing of the computational burdens).

### Example Workflow for Generating Mappings and Training Models to Suggest Resident Services

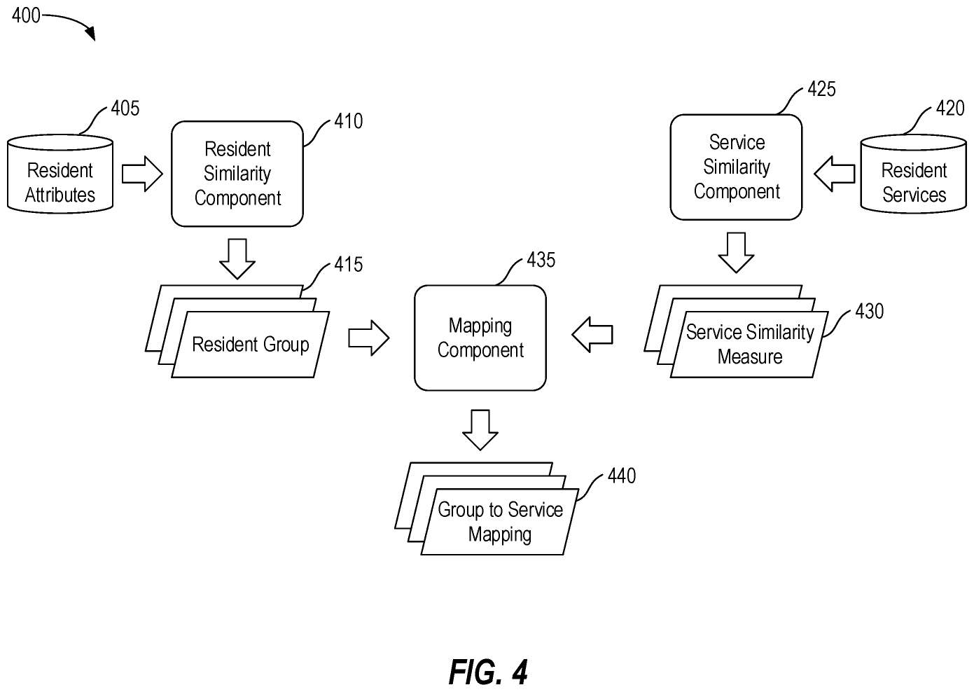

FIG. 4 depicts an example workflow 400 for generating mappings and training models to suggest resident services. In some aspects, the workflow 400 is performed by a machine learning system, such as the machine learning system 135 of FIG. 1 and/or the machine learning system 335 of FIG. 3.

In the illustrated example, the workflow 400 generally includes three broad operations, including a service-to-service similarity operation (e.g., performed by the service similarity component 425), a resident-to-resident similarity operation (e.g., performed by the resident similarity component 410), and a resident group-to-services mapping operation (e.g., performed by a mapping component 435). Though three discrete components are depicted for conceptual clarity, in embodiments, the operations involved in the workflow 400 may be combined or distributed across any number of components, and may be implemented using hardware, software, or a combination of hardware and software. In some aspects, the workflow 400 depicts a collaborative filtering approach to service plan generation.

In the illustrated workflow, a set of resident attributes 405 are accessed for processing by a resident similarity component 410. In an aspect, the resident similarity component 410 seeks to determine or quantify resident similarity according to a variety of data included in the resident attributes 405. In some embodiments, the resident similarity component 410 calculates or quantifies the resident similarities based on multiple criteria or features, such as demographic data (e.g., gender, age, and the like), any indicated medical problems (e.g., diagnoses, allergies, and the like), any medications the resident consumes, and the like. This can significantly improve model accuracy over conventional collaborative filtering approaches that typically compare users using a single feature or attribute. In some embodiments, to generate the resident-to-resident similarity measure for each pair of residents in the resident attributes 405, the resident similarity component 410 can map the resident attributes 405 in a multidimensional space, and compute the distance between each pair of residents.

In the illustrated embodiment, the resident similarity component 410 can further use the generated resident-to-resident similarity measures across multiple features to generate clusters of similar residents (e.g., resident groups 415). In an embodiment, each resident group 415 corresponds to a set of values (or ranges of values) for the various features in the resident attributes 405, such that each resident group 415 defines a logical group or cluster of residents. In this way, current and future residents can be assigned to a given resident group 415 based on their own attributes. In at least one embodiment, for a given resident to be assigned to a given group (e.g., during inferencing to generate a service plan), the machine learning system can determine whether the new resident shows feature similarity (e.g., a value that is within a defined distance from a value, or is included in a defined range of values, associated with the group) across one or more of the defined features.

For example, if the age range associated with a given resident group 415 is 50-60 years, the system may determine whether the new resident is within that range. Similarly, if the age value associated with a given resident group 415 is 55, the system may determine whether the new resident is within a defined threshold to that value (or is closest to that value, as compared to the age value of each other resident group 415). If so, the age feature may be considered or labeled as a match. In some embodiments, to assign a new resident to a given resident group 415, the system determines whether the resident shows similarity/matches at least a defined portion or percentage of the features/attributes. For example, the system may determine whether the new resident matches or is similar to at least N % of the features or attributes used to define the feature groups 415, where N may be a configurable or defined value set by a user.

As an example, suppose there are two new residents: a first resident who is a female in the 52-57 age range who has cancer and undergone surgery, and a second resident who is a female in the same age range who is receiving radiation therapy. As both residents belong to the same age group, gender, and diagnosis, if the threshold similarity is set at one level (e.g., 50%), they may be placed in the same category or resident group 415. However, if the threshold is set at greater threshold (e.g., 75%), even though they have similar conditions and demographic features, the residents may still be assigned to different resident groups 415 due to the differing medical treatment(s) or other attributes.

In this way, while training or generating the service model, the machine learning system can generate or define resident groups 415 based on similarity, which can make future inferencing more computationally efficient and accurate.

In the illustrated example, a set of resident services 420 are accessed for processing and evaluation by a service similarity component 425. In one embodiment, the service similarity component 425 is used to determine or identify similar services, from the set of resident services 420, such as by identifying how similar the services are to one another based on the combinations in which they occur or are used (e.g., which combinations of services are often used together for the same resident(s)). In an embodiment, the resident services 420 include sets of services, one set for each resident, where each set indicates the services that are provided to the corresponding resident.

In at least one embodiment, to determine service similarity, the service similarity component 425 can use these sets of services provided to the residents as inputs to a static model, such as a matrix quantifying service or operation, to derive service-to-service similarity measures. These similarities may be determined or generated, for example, based on how frequently various services combinations occur in the resident services 420. That is, the service similarity measures 430 may be generated based on determining, for each respective pair of services, how often the services are used in combination with each other for residents (as reflected in the resident services 420).

For example, if bathroom assistance services was provided in combination with walking assistance services five times in the resident services 420 (e.g., five residents received both bathroom assistance and walking assistance), in combination with dressing and/or grooming assistance services eight times in the resident services 420 (e.g., eight residents received both bathroom assistance and dressing and grooming assistance), and in combination with dining assistance services three times in the resident services 420 (e.g., three residents received both bathroom assistance and dining assistance), then the service similarity measures 430 may indicate that the bathroom assistance service is more similar to a dressing and grooming service than it is to walking assistance services and/or dining assistance services (as well as that bathroom assistance is more similar to walking assistance than to dining assistance).

In this way, while training or generating the service model, the machine learning system can generate or define service similarity measures 430 based on service co-occurrence, which can make future inferencing more computationally efficient and accurate.

In the illustrated example, the resident groups 415 and service similarity measures 430 are then accessed for processing by a mapping component 435 to generate a set of group-to-service mappings 440. In an embodiment, the mapping component 435 can generate the group-to-service mappings 440 by identifying, for each resident group 415, a set of services (from the resident services 420) used by one or more residents included in the resident group 415. Using the service similarity measures 430, the mapping component 435 can then identify other service(s), not included in the originally-identified set of services for the resident group 415, that are similar to the original set of services (e.g., that have a service similarity measure 430 that meets or exceeds a defined threshold for one or more of the services in the set of services associated with the resident group 415).

Stated differently, for each respective resident group 415, the mapping component 435 can first identify a set of services that one or more of the residents in the group used or received. For each such service in this set, the mapping component 435 can then identify one or more other services, based on the service similarity measures 430, that are sufficiently-similar. In this way, the mapping component 435 can expand the set of potential services to include others not-otherwise associated with the specific resident group 415, which can substantially improve the accuracy of the service suggestions, as well as the computational efficiency of the system.

Using this learned group-to-service mapping 440, the machine learning system can find or identify services which were being provided to each particular group of residents having similar medical backgrounds or attributes. For instance, if a first resident has chronic pain, takes psychiatric drugs, and frequently requires walking assistance services, it may be likely that a second resident using comparable prescriptions for chronic pain will also require such walking services. For a third resident, who similarly experiences chronic pain but does not use any psychiatric medications, the walking services may not be relevant or needed.

As discussed below in more detail, with this model in place, when a new resident is admitted to a facility (or when a service plan is otherwise being generated for a current resident), information about the resident's demographics, medical history (such as diagnoses and allergies) and medications can be entered into the system in real-time, allowing the learned model to identify the resident group to which the new resident most closely aligns, and suggest the relevant services. These services may further be scored or ranked based on fitness scores, as discussed in more detail below.

### Example Workflow for Generating Suggested Service Plans

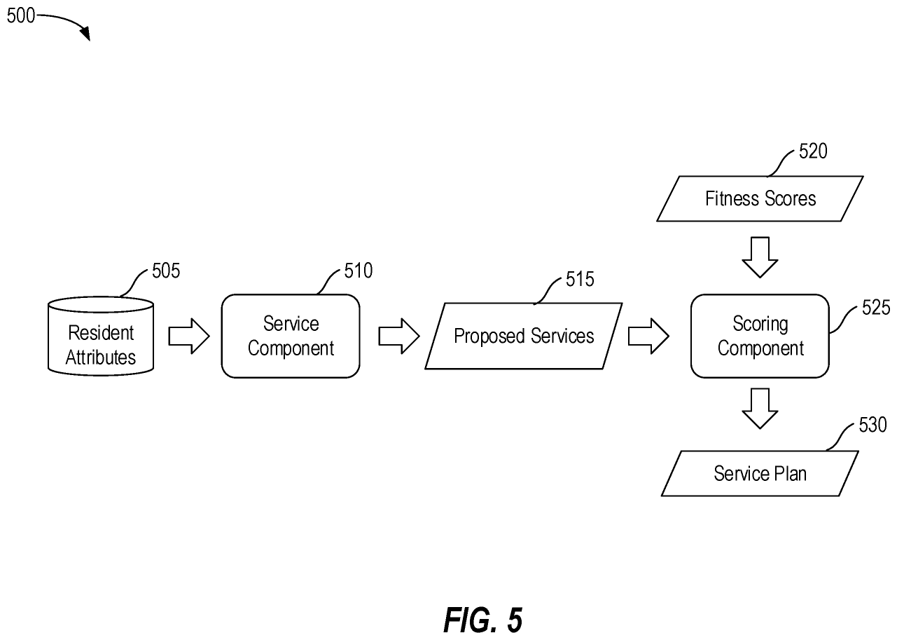

FIG. 5 depicts an example workflow 500 for generating suggested service plans. In some aspects, the workflow 500 is performed by a machine learning system, such as the machine learning system 135 of FIG. 1 and/or the machine learning system 335 of FIG. 3. In some embodiments, the workflow 500 is performed using the model trained/generated using the workflow 400 of FIG. 4.

In the illustrated workflow 500, a service component 510 and a scoring component 525 are used to generate and evaluate service plans 530. Though these discrete components are depicted for conceptual clarity, in embodiments, the operations involved in the workflow 500 may be combined or distributed across any number of components, and may be implemented using hardware, software, or a combination of hardware and software.

In the illustrated example, a set of resident attributes 505 are received for processing by a service component 510. As discussed above, the resident attributes 505 can generally include characteristics or information for a resident of a residential facility, which may include a new resident being admitted, a current resident for whom a new service plan is being created, and the like. The resident data 505 can generally include a variety of data or features, as discussed above, such as the resident's demographics, diagnoses, medications, and the like.

As illustrated, the service component 510 evaluates the resident data 505 to generate a set of proposed services 515. In some aspects, as discussed above, the service component 510 can evaluate the resident attributes 505 to assign the resident to a group or cluster of residents (e.g., a resident group 415 of FIG. 4), which were learned during training of the model, and where the assignment is performed based on attribute similarity. In some embodiments, the service component 510 can assign the resident to a single resident group by finding the group with which the resident shares the most number of features or attributes. In at least one embodiment, the service component 510 can assign the resident to multiple resident groups (e.g., all groups where the resident matches N % or more of the features).

As discussed above, the service component 510 can then generate the set of proposed services 515 (also referred to as potential services or alternative services in some aspects) using a mapping (e.g., group-to-service mapping 440 of FIG. 4) learned as part of training the service plan model. For example, as discussed above, the mapping may indicate which service(s) or set(s) of services are potentially relevant for residents associated with the assigned resident group(s).

In the illustrated example, these proposed services 515 are accessed and evaluated by a scoring component 525 based on a set of fitness scores 520. As discussed above, the fitness scores 520 may be generated by evaluating a set of historical data to determine how appropriate or suitable a given service was for a given resident, based on the resident's attributes. In some aspects, therefore, these fitness scores 520 can be used to determine how suitable or appropriate each service in the proposed services 515 is, with respect to the new resident, based on the resident attributes 505.

In some aspects, the scoring component 525 uses the fitness scores 520 to generate a numerical fitness score or other measure for each service in the set of proposed services 515. The scoring component 525 (or another component) may then sort and/or filter the proposed services 515 based on these scores. For example, in some aspects, the scoring component 525 can filter out any proposed services with a score below a defined threshold. In some embodiments, the scoring component 525 can output the proposed services, along with the generated set of predicted fitness scores, via a graphical user interface (GUI) for a user to review. For example, the scoring component 525 may generate a visualization to enable the user to rapidly and efficiently identify an optimal set of services, such as by adjusting the color, size, emphasis, or other attribute of the visualization for each proposed service 515 in order to highlight the most-suitable set.

In the illustrated example, the scoring component 525 (or a user) can thereby generate a service plan 530. In some embodiments, the service plan 530 can include a subset of the proposed services 515, selected based at least in part on predicted fitness scores (generated based on the fitness scores 520). In some embodiments, as discussed above, the system may then automatically engage or implement the service plan 530, may output it for users to implement, and the like.

### Example Method for Training Machine Learning Models to Generate Service Plans

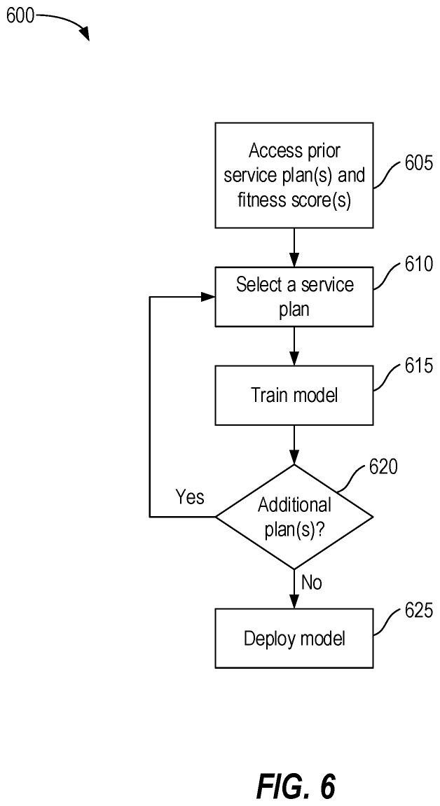

FIG. 6 depicts a flow diagram depicting an example method 600 for training machine learning models to generate service plans. In some embodiments, the method 600 is performed by a machine learning system, such as the machine learning system 135 of FIG. 1, the machine learning system 335 of FIG. 3, and the like.

At block 605, the machine learning system accesses or receives a set of prior service plans and fitness scores. As discussed above, the prior service plans (e.g., service plans 130 of FIG. 1) can generally indicate, for one or more current or past residents of one or more residential facilities, a corresponding set of services that were provided to the resident for at least some period of time. In some embodiments, each service plan can further include or indicate the attributes of the corresponding resident (e.g., their demographics, diagnoses, and the like). In an embodiment, the fitness scores (e.g., fitness scores 240 of FIG. 2) may be generated based on a variety of features of the service (such as natural language notes) to indicate the fitness or suitability of each service for a given resident, based on the resident's attributes. Collectively, the prior service plans, resident attributes, and/or fitness scores may be referred to in some aspects as training data or training exemplars.

At block 610, the machine learning system selects a service plan from the set of prior plans, along with corresponding fitness score(s) indicating how suitable or appropriate the service plan was for the resident. That is the machine learning system selects a training exemplar. Generally, the machine learning system may select the service plan using any suitable technique, including randomly or pseudo-randomly, as all available prior plans will be used to train the model using the method 600. Although the illustrated example depicts a sequential process to iteratively select and evaluate each service plan in turn (e.g., to train a model using stochastic gradient descent), in some aspects, the machine learning system can process some or all of the prior plans in parallel (e.g., to train the model using batch gradient descent or collaborative filtering).

At block 615, the machine learning system trains a machine learning model based on the selected service plan(s). For example, as discussed above, the fitness scores for each service in the service plan may be used as the target output of the model, based on input resident attributes. In at least one embodiment, the machine learning system uses a modified collaborative filtering approach to train the service plan model, as discussed above and in more detail below. In this way, the model learns to generate service plans (or to generate predicted scores for services and/or service plans), allowing new residents to be admitted and processed using accurate, objective, and computationally-efficient techniques.

At block 620, the machine learning system determines whether there is at least one additional prior service plan that has not-yet been used to train the model. If so, the method 600 returns to block 610. If not, the method 600 continues to block 625, where the machine learning system deploys the trained model for inferencing during runtime. For example, the machine learning system may transmit or otherwise provide the model to one or more downstream systems (e.g., computing systems at residential facilities), or may itself use the trained model to generate service plans.

### Example Method for Extracting Features to Drive Model Training for Service Plan Generation

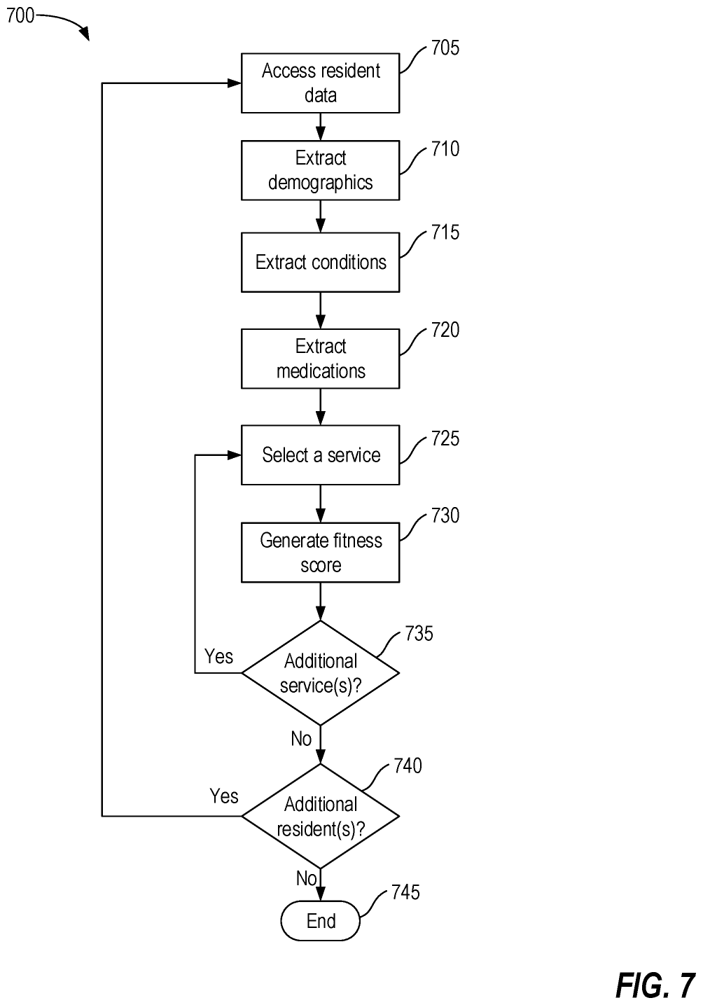

FIG. 7 depicts a flow diagram depicting an example method 700 for extracting features to drive model training for service plan generation. In some embodiments, the method 700 is performed by a machine learning system, such as the machine learning system 135 of FIG. 1, the machine learning system 335 of FIG. 3, and the like.

In some aspects, the method 700 provides additional detail for techniques to generate training samples or exemplars to train machine learning models, as discussed above. For example, the method 700 may be used to process historical data in order to extract and/or transform the relevant features, generate fitness scores, and the like. These training exemplars can then be used to train one or more machine learning models.

At block 705, the machine learning system accesses resident data to be used to train a model (e.g., resident attributes 110 of FIG. 1). As discussed above, the resident data generally includes data describing one or more residents of one or more residential care facilities for one or more previous points in time. For example, the resident data may indicate, for each resident, their demographics (e.g., age, sex, and the like), any diagnoses or conditions they have, any medications they take, and the like. In the illustrated example, at block 705, the machine learning system accesses the resident data for a single resident. Although the illustrated example depicts a sequential process (accessing and evaluating each resident's data in turn) for conceptual clarity, in aspects, the machine learning system can process some or all of the residents in parallel.

At block 710, the machine learning system extracts the demographic(s) from the resident data. As discussed above, the resident demographics used by the machine learning system can generally include a variety of features or attributes depending on the particular implementation. For example, the resident demographics may include their sex, race, marital status, age, and the like.

At block 715, the machine learning system extracts the condition(s) specified in the resident data. As discussed above, the resident conditions (also referred to as diagnoses) used by the machine learning system can generally include a variety of features or attributes depending on the particular implementation. For example, the extracted conditions may include indications as to whether the resident has any specific diagnoses (e.g., from a defined list of diagnoses), the reported severity of any such conditions or diagnoses, how long the resident has had the diagnoses or conditions, and the like.

At block 720, the machine learning system extracts the medication(s) specified in the resident data. As discussed above, the resident medications used by the machine learning system can generally include a variety of features or attributes depending on the particular implementation. For example, the extracted medications may include indications as to whether the resident receives or uses any specified medications (e.g., from a defined list of medications or medication classes), the dosage of such medications (e.g., the amount and/or frequency of administration), the type or method of administering the medications (e.g., orally, via IV, and the like), and the like.

At block 725, the machine learning system selects one of the services, indicated in the resident data, that was provided to the user. In an aspect, the machine learning system can use any suitable technique to select the service, including random or pseudo-random selection, as the machine learning system will evaluate each service in turn. Although the illustrated example depicts a sequential process (evaluating each service in turn) for conceptual clarity, in some embodiments, the machine learning system can process some or all of the services in parallel.

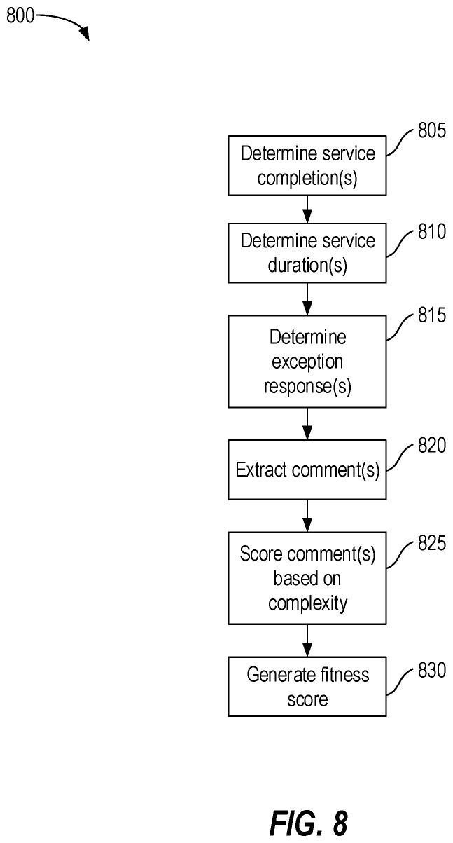

At block 730, the machine learning system generates a fitness score for the selected service, as discussed above. The fitness score generally indicates the suitability or appropriateness of the selected service for the specific resident (e.g., a measure or score indicating the degree to which the resident needed, relied on, or benefitted from the service). One example method for generating the fitness score is discussed in more detail below with reference to FIG. 8. In this way, the fitness score(s) can be used as target variables for the input resident attributes (e.g., demographics, conditions, and medications).

At block 735, the machine learning system determines whether there is at least one additional service, received by the resident, which has not-yet been evaluated and scored. If so, the method 700 returns to block 725. If not, the method 700 continues to block 740. At block 740, the machine learning system determines whether there is at least one additional resident and/or one additional historical service plan that has not-yet been evaluated to generate a training exemplar. If so, the method 700 returns to block 705. If not, the method 700 terminates at block 745. In this way, the machine learning system can generate training exemplars for use in training machine learning models to predict service fitness scores and generate service plans.

### Example Method for Scoring Historical Services to Generate Training Data for Machine Learning Models

FIG. 8 is a flow diagram depicting an example method 800 for scoring historical services to generate training data for machine learning models. In some embodiments, the method 800 is performed by a machine learning system, such as the machine learning system 135 of FIG. 1, the machine learning system 335 of FIG. 3, and the like.

In the illustrated example, the method 800 can be used to generate a fitness score for a single instance or performance of a service. For example, the method 800 may be used to generate a fitness score indicating the suitability of the service and/or probability that the resident needed the service on a specific day and/or at a specific time. In some embodiments, the machine learning system can generate fitness scores for each such service performance, and aggregate these scores (e.g., by finding the sum or average score) for each performance of a single service in order to generate an overall suitability or fitness score for the service, with respect to the user. For example, the machine learning system may generate a first fitness score based on the caregiver providing the service on one day, a second fitness score for providing the service on the next day, and so on. In other embodiments, the machine learning system may process information from multiple service performances in parallel to generate an overall score.

At block 805, the machine learning system determines one or more service completion statuses associated with providing a prior service. In some embodiments, as discussed above, the completion status can be reflected as a flag or other label (e.g., added by a user such as a caregiver) to indicate the completion status of the service. For example, as discussed above, the machine learning system can determine whether the service was completed, not completed, completed with one or more exceptions, and the like. In some embodiments, each completion status may have a corresponding defined value that can be used to generate the fitness score. For example, “completed” statuses may have a higher value to result in a higher fitness score. As discussed above, the completion status can thereby be used to generate or determine a subscore that is used to generate the fitness score.

At block 810, the machine learning system determines the service duration(s) associated with providing the service. For example, the machine learning system may determine the number of minutes that the caregiver spent performing the service. In some embodiments, the machine learning system can generate a duration subscore by modifying or transforming the total duration. For example, if the duration indicates the number of minutes that the caregiver used to provide the service, the machine learning system may divide this duration by a predefined value to generate the subscore.

At block 815, the machine learning system determines zero or more exception responses associated with providing the prior service. In some embodiments, as discussed above, the exception response can be reflected as a flag or other label (e.g., added by a user such as a caregiver) to indicate if any exceptions or difficulties arose when providing the service. For example, as discussed above, the machine learning system can determine whether the resident was sick, the resident refused the service, the resident was absent, the resident required more assistance, the service required an additional assistive device, and the like. In some embodiments, each exception response may have a corresponding defined value that can be used to generate the fitness score. For example, an exception status of “additional assistance needed” may have a higher value to result in a higher fitness score, as compared to a status of “no exceptions.” As discussed above, the exception responses can thereby be used to generate or determine a subscore that is used to generate the fitness score.

At block 820, the machine learning system extracts any natural language comments associated with providing the prior service. For example, as discussed above, the caregiver that provided the service may type, handwrite, or record themselves to describe providing the service and/or to describe the resident, such as to indicate how agreeable the resident was, to indicate any difficulties or problems that arose, and the like.

At block 825, the machine learning system can then score the extracted comment(s) based on their complexity. For example, as discussed above, a sentiment model may be used to process the natural language text in order to generate a measure or score indicating how complex the comment is and/or how complex the service performance was, as discussed above. In an embodiment, the fitness score may be directly related to this complexity score, such that more complexity results in a higher fitness score (indicating that the service is likely needed).

At block 830, the machine learning system can then generate a fitness score for the service based on the subscores determined above. In an embodiment, as discussed above, the machine learning system can generate the fitness score by aggregating these subscores, such as using a weighted average (where the weights for each subscore may be learned or user-specified). In some embodiments, the machine learning system can further scale or normalize the aggregate (e.g., by dividing the generated score by the maximum possible score, or by scaling the score to a value between zero and one). This can allow for more efficient and accurate understandings of the service suitability.

In some embodiments, as discussed above, the machine learning system can use the method 800 to generate a respective fitness score for each respective time the service was performed or provided to the resident. In one such embodiment, the machine learning system can then aggregate the fitness scores over time (e.g., by averaging them) to generate an overall fitness score for the service with respect to the resident.

In another embodiment, the machine learning system may process data from multiple instances of providing the service to generate each subscore. For example, the machine learning system may generate a completion status subscore based on multiple visits (e.g., based on the percentage or proportion of the time that the service was successfully completed). Similarly, a duration subscore may be generated based on the average or total time spent across multiple service performances, and an exception subscore may be generated based on data such as the average exception response or the percentage of times that the exception response meets or exceeds a defined threshold level of complexity. Further, the comments subscore may be generated based on multiple comments, such as the average complexity of the comments, the number or proportion of the time that the comment complexity exceeds a threshold, and the like. These aggregated subscores can then be used to generate an overall fitness score in some aspects.

As discussed above, the fitness score(s) can then be used as target variables to improve training of one or more machine learning models. In this way, the machine learning system can learn how appropriate or suitable any given service is likely to be for a given resident (e.g., the probability that the resident needs or will benefit from the service) based on the resident's individual attributes. This substantially improves computational efficiency of generating service plans, and further reduces uncertainty and improves resident outcomes.

### Example Method for Generating Mappings for Improved Machine Learning to Generate Service Plans

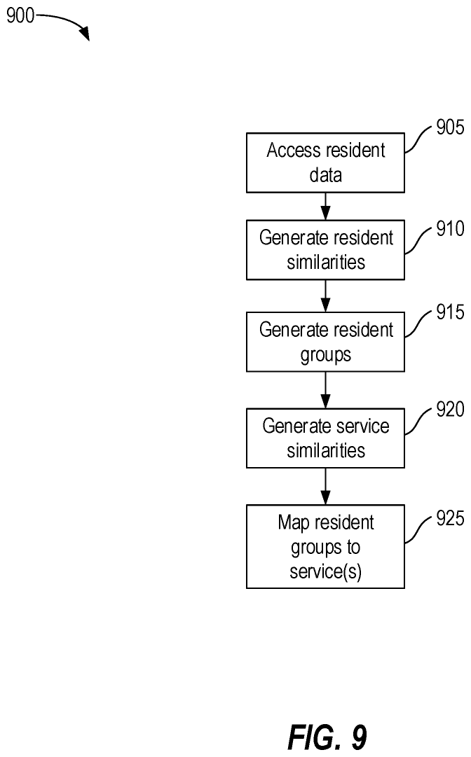

FIG. 9 is a flow diagram depicting an example method 900 for generating mappings for improved machine learning to generate service plans. In some embodiments, the method 900 is performed by a machine learning system, such as the machine learning system 135 of FIG. 1, the machine learning system 335 of FIG. 3, and the like. In one embodiment, the method 900 provides additional detail for the workflow 400 of FIG. 4. In one embodiment, the method 900 provides additional detail for block 615 of FIG. 6.

At block 905, the machine learning system accesses historical resident data. As discussed above, the resident data can generally indicate one or more attributes of residents in a residential facility, one or more service(s) provided to the residents, and the like. For example, the resident data may correspond to the resident attributes 405 and/or the resident services 420 of FIG. 4.

At block 910, the machine learning system generates resident similarities based on the resident data. For example, based on resident attributes for each resident in the resident data, the machine learning system may generate a set of pairwise resident-to-resident similarities indicating, for each given resident, how similar the given resident is to each other resident. In some embodiments, this resident similarity is performed using multiple resident attributes, such as demographics, diagnoses, medications, and the like, as compared to using a single resident feature to define similarity. In some embodiments, the resident similarities are generated by a resident similarity component, such as the resident similarity component 410 of FIG. 4.

At block 915, the machine learning system generates a set of resident groups (e.g., resident groups 415 of FIG. 4) based on the resident similarities. For example, in one aspect, the machine learning system can generate resident groups by grouping or clustering residents having similar attributes and/or high resident similarities. That is, the resident similarity across multiple features or attributes can be used to generate resident groups, where each group is associated with a corresponding set of values (or ranges of values) for each of the features or attributes. In this way, as discussed above, new residents can be sorted or classified into one or more resident groups based on their attributes.

At block 920, the machine learning system generates a set of service similarities (e.g., service similarity measures 430 of FIG. 4) based on the resident data. For example, based on combinations of services provided to the residents in the resident data, the machine learning system may generate a set of pairwise service-to-service similarities indicating, for each given service, how similar the given service is to each other service. In some embodiments, this service similarity is generated based on how frequently or often the service(s) are provided in combination (e.g., how often the services are both provided to the same resident). In some embodiments, the service similarities are generated by a service similarity component, such as the service similarity component 425 of FIG. 4.

At block 925, the machine learning system can then map the resident groups (generated at block 915) to services based on the service(s) used by each resident, as well as the service similarities (generated at block 920). For example, in one embodiment, for each resident group, the machine learning system can identify the service(s) that were provided to one or more residents in the group (based on the resident data). For each such service, the machine learning system may then use the service similarity measures to identify one or more other services that are similar to the provided service. In this way, the machine learning system can combine resident-to-resident similarity with service-to-service similarity to generate more accurate and reliable service suggestions in a more computationally efficient manner.

In an embodiment, as discussed above, the trained model (e.g., the learned groupings and mappings) can then be used to generate new service plans for residents based on their individual attributes.

### Example Method for Generating Service Plans Using Machine Learning

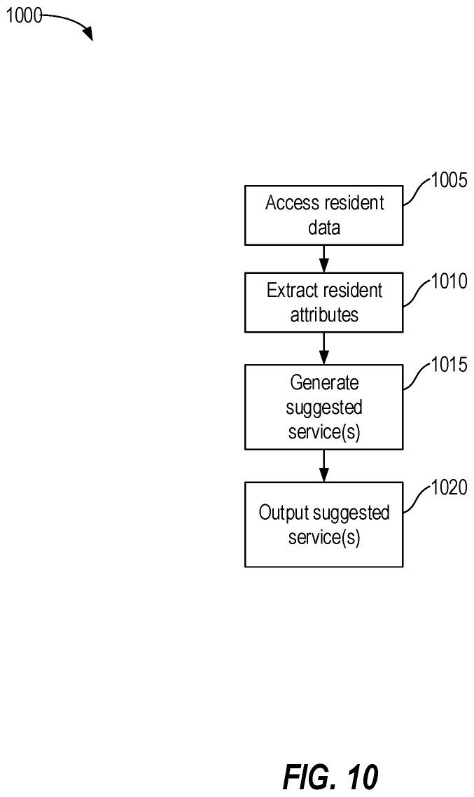

FIG. 10 is a flow diagram depicting an example method 1000 for generating service plans using machine learning. In some embodiments, the method 1000 is performed by a machine learning system, such as the machine learning system 135 of FIG. 1, the machine learning system 335 of FIG. 3, and the like.

At block 1005, the machine learning system accesses resident data for a new, current, or future resident of a residential care facility. For example, the resident data may include information relating to a current resident for whom a new service plan is being generated (e.g., due to changed or new attributes, such as a new medication or diagnosis), information relating to a prospective resident that is enrolling or being admitted to the residential care facility (e.g., where no service plan exists), and the like. In some aspects, the resident data corresponds to the resident data 305 of FIG. 3.

At block 1010, the machine learning system extracts one or more resident attributes from the resident data. As discussed above, the machine learning system can generally extract and/or preprocess the attributes related to specific features used by the model, such as the resident's demographics, conditions, medications, and the like. In embodiments, the specific attributes or features extracted, as well as the specific preprocessing applied to them, can vary depending on the particular implementation.

At block 1015, the machine learning system generates a set of suggested or proposed services for the resident by processing the extracted attributes using a machine learning model, as discussed above. Generally, the process of generating the set of suggested services may vary depending on the particular implementation and model architecture. For example, in the case of a neural network, the machine learning system may use the attributes as input to generate a set of scores, each score indicating the predicted suitability or necessity of a corresponding service for the resident. In the case of a collaborative filtering model, the machine learning system may use the attributes to identify a resident group, map this group to specific services, and/or score each service, as discussed above.

At block 1020, the machine learning system outputs the suggested services, such as via a GUI. For example, as discussed below in more detail, the machine learning system may dynamically update a visualization on the GUI to depict the various potential services, the predicted suitability of each (e.g., the probability that each service will be useful, needed, or otherwise appropriate or suitable for the resident), and the like.

In some embodiments, outputting the suggested services includes displaying them or otherwise providing them to a user (e.g., a caregiver), and requesting review and/or approval. In some embodiments, outputting the services includes implementing them, such as by scheduling or adding one or more services to the resident's profile or calendar. In this way, the machine learning system can efficiently and accurately generate service suggestions for residents.

### Example Method for Scoring and Ranking Services Using Trained Models

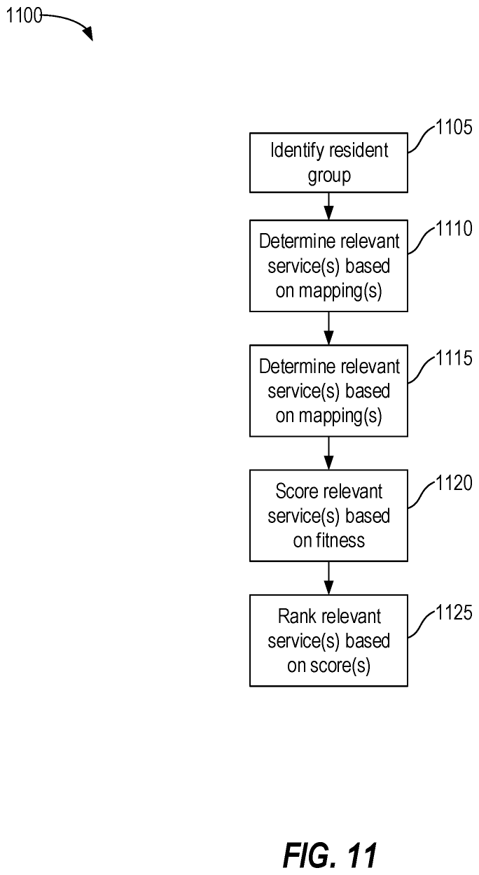

FIG. 11 is a flow diagram depicting an example method 1100 for scoring and ranking services using trained models. In some embodiments, the method 1100 is performed by a machine learning system, such as the machine learning system 135 of FIG. 1, the machine learning system 335 of FIG. 3, and the like. In one embodiment, the method 1100 provides additional detail for the workflow 500 of FIG. 5, and/or for block 1015 of FIG. 10.

At block 1105, the machine learning system identifies a resident group to assign to the resident (for whom a service plan is being created), based on resident attributes. For example, as discussed above, the machine learning system may identify which resident group(s) the resident is most-closely aligned with, which groups match the resident for at least a threshold number or percentage of features or attributes, and the like.

At block 1110, the machine learning system determines a set of relevant or potential services for the resident based on a set of resident group to service mappings. For example, as discussed above, the machine learning system may access and evaluate mappings such as the group to service mappings 440 of FIG. 4 to identify a set of services that may be appropriate or useful for the new or current resident.

At block 1115, the machine learning system can generate a score (e.g., a predicted fitness score) for each service in the set of potential services based on generated fitness scores for prior residents/services. For example, as discussed above, the fitness scores may indicate the suitability or probability that the given service is useful to or needed by one or more given residents based on their attributes. Using these scores, the machine learning system may generate a predicted fitness score that indicates the probability that the specific service will be useful for the new/current resident.

At block 1120, the machine learning system can then rank, sort, and/or filter the set of relevant potential services based on the generated score(s). For example, as discussed above, the machine learning system may filter the services to remove any with a score below a defined threshold, sort the services based on the scores, and the like. In some embodiments, the machine learning system can suggest a subset of the potential services having a minimum predicted score. In other embodiments, the machine learning system can output all of the potential services, along with the corresponding scores, and allow a user (e.g., a caregiver) to select among them.

### Example Method for Updating Graphical User Interfaces Based on Machine Learning Models

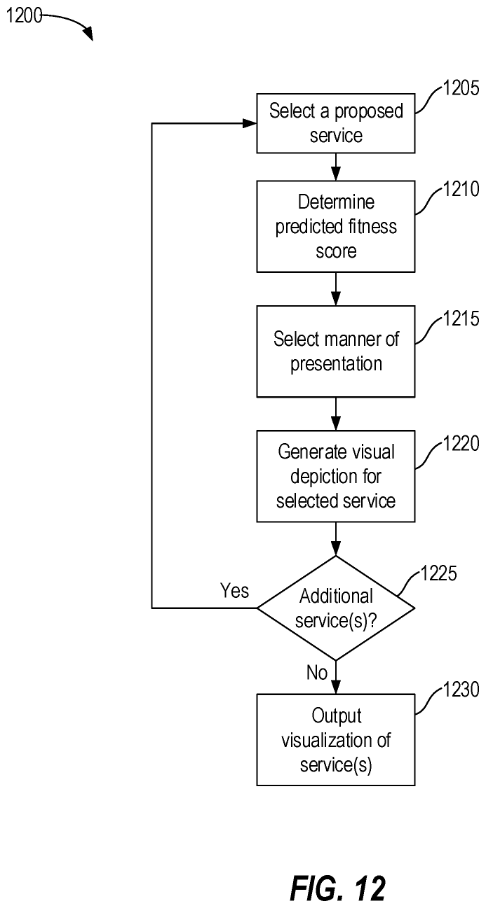

FIG. 12 is a flow diagram depicting an example method 1200 for updating graphical user interfaces (GUIs) based on machine learning model output for service plan generation. In some embodiments, the method 1200 is performed by a machine learning system, such as the machine learning system 135 of FIG. 1, the machine learning system 335 of FIG. 3, and the like. In one embodiment, the method 1200 provides additional detail for block 1020 of FIG. 10.

At block 1205, the machine learning system selects a proposed service, from a set of proposed services generated for the service plan. As discussed above, in one embodiment, the machine learning system can generate a set of proposed services by processing resident attributes for a new resident using one or more trained machine learning models. In an embodiment, the machine learning system may select the proposed service using any suitable technique, including randomly or pseudo-randomly, as the machine learning system will select each proposed service in turn. Although the illustrated example depicts a sequential process (iteratively evaluating each service in turn) for conceptual clarity, in embodiments, the machine learning system may process some or all of the potential services in parallel.

At block 1210, the machine learning system determines the predicted fitness score that was generated for the selected service. As discussed above, the predicted fitness score can generally indicate the suitability or appropriateness of the service for the specific resident. For example, the predicted fitness score may indicate the probability that the resident needs, will benefit from, or otherwise should receive the service. In some embodiments, the predicted fitness scores are numerical values in a defined range (e.g., between zero and one or between zero in ten), where higher values indicate a higher suitability or appropriateness of the service for the resident.

At block 1215, the machine learning system selects a manner of presentation for the selected service based on the corresponding predicted fitness score. Generally, the manner of presentation may be selected in a variety of ways and may include a variety of presentation techniques, depending on the particular implementation. In some embodiments, the machine learning system selects the manner of presentation based on defined mappings or rules in order to dynamically emphasize and deemphasize service alternatives based, at least in part, on their predicted fitness scores.

For example, the machine learning system may select a color to use to represent the service (e.g., where colors such as green indicate higher fitness scores, while colors such as red indicate lower fitness scores), a size of the visual element used to represent the service (e.g., the height of a bar or other visual element that represents the service, the size of the font, and the like), an ordering of the services (e.g., where services with higher scores are presented nearer to the top of the interface), as well as any other visualization (e.g., determining to highlight or make bold any services with a fitness score that meets or exceeds a threshold, to hide or remove any services with a fitness score below a threshold, and the like).

At block 1220, the machine learning system then generates a visual depiction, (e.g., to be output on a GUI) to represent the selected service based on the selected manner of presentation.

At block 1225, the machine learning system determines whether there is at least one additional service, in the set of proposed or potential services, that has not-yet been evaluated. If so, the method 1200 returns to block 1205. If not, the method 1200 continues to block 1230, where the machine learning system outputs the generated visualization, including one or more of the set of proposed services and fitness scores, via a GUI. In this way, complex machine learning model evaluations and output can be readily visualized in an efficient and dynamic manner using GUIs to present the data in an unconventional way that improves the functioning of the interface and the computing device, as well as improving the overall functionality of the system and the residential care facility.

### Example Method for Generating Facility-Wide Service Plan Data Using Machine Learning

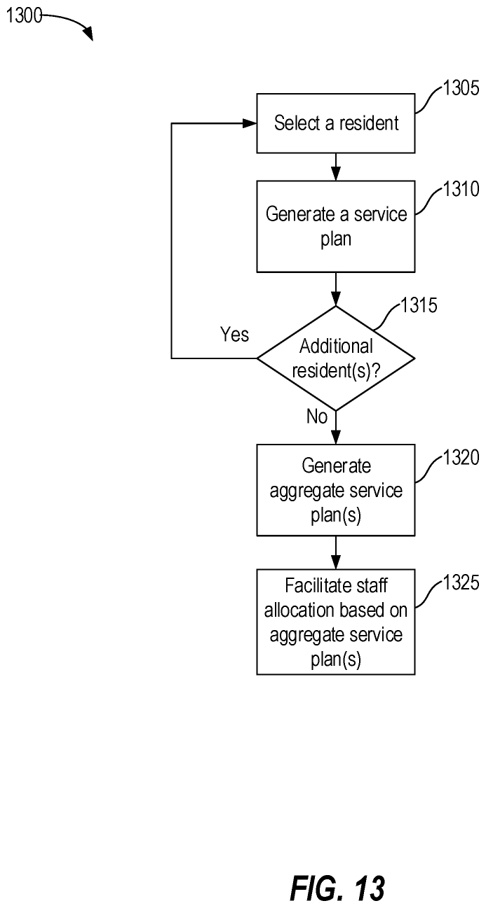

FIG. 13 is a flow diagram depicting an example method 1300 for generating facility-wide service plan data using machine learning. In some embodiments, the method 1300 is performed by a machine learning system, such as the machine learning system 135 of FIG. 1, the machine learning system 335 of FIG. 3, and the like.

At block 1305, the machine learning system selects a current resident of a residential facility. In some aspects, the machine learning system selects a resident of a single facility, enabling the machine learning system to generate aggregated information for the single facility. Generally, the machine learning system can select the resident using any suitable criteria, including randomly or pseudo-randomly, as all residents of the facility can be evaluated using the method 1300. Although the illustrated example depicts a sequential process (e.g., selecting and evaluating each resident in turn) for conceptual clarity, in embodiments, the machine learning system may process some or all of the residents in parallel.

At block 1310, the machine learning system generates a service plan for the selected resident using one or more machine learning models. For example, as discussed above with reference to FIGS. 3, 5, 10, and 11, the machine learning system may use trained machine learning models to process resident attributes in order to generate and/or score a set of services based on how the probability that the resident needs or will benefit from the service. In an embodiment, block 1310 may include generating a new service plan for the resident (e.g., for a new resident, or based on new data for an existing resident) as well as accessing or retrieving a prior-generated service plan for a current resident.

At block 1315, the machine learning system determines whether there is at least one additional resident that has not-yet been evaluated. If so, the method 1300 returns to block 1305. If not, the method 1300 continues to block 1320, where the machine learning system generates one or more aggregated service plans for the facility based on the service plans generated for each individual resident.

In some embodiments, the aggregated service plan can indicate information such as the overall set of services used by any resident of the facility, the number of resident(s) that receive each specific service, the overall complexity of each service with respect to the facility, the number of staff members that are needed to fulfill the aggregated service plan, and the like.

At block 1320, the machine learning system can then facilitate staff allocation(s) for the facility based on the aggregated service plan(s). For example, based on the specific set of services that each resident uses, the machine learning system can identify the number and/or mix of staff that will be needed to fulfill the services, and suggest or allocate such staff for the facility.

In this way, the system can improve the operations of the residential care facility and generally improve resident outcomes while reducing expense and subjectivity of the service planning process.

### Example Method for Training Machine Learning Models to Generate Residential Service Plans

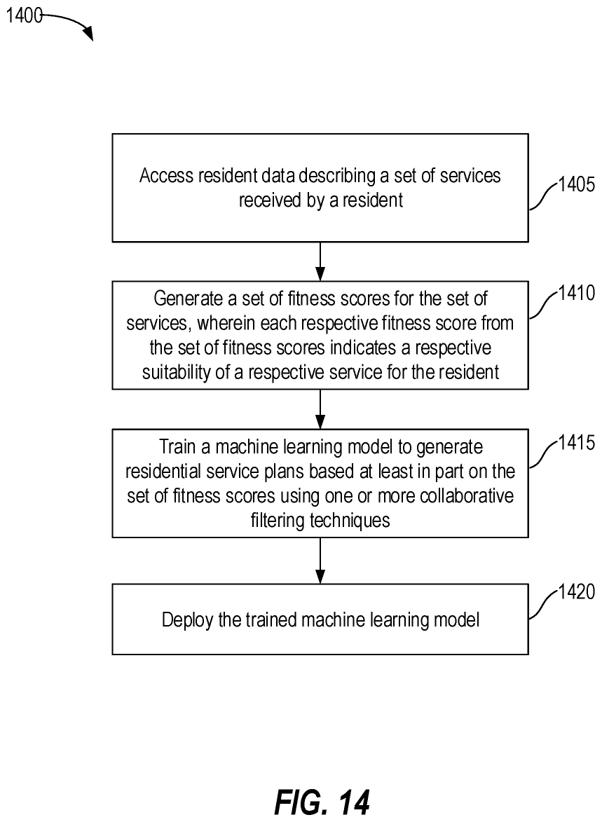

FIG. 14 is a flow diagram depicting an example method 1400 for training machine learning models to generate residential service plans. In some embodiments, the method 1400 is performed by a machine learning system, such as the machine learning system 135 of FIG. 1, the machine learning system 335 of FIG. 3, and the like.

At block 1405, resident data (e.g., historical data 105 of FIG. 1) describing a set of services received by a resident is accessed.

At block 1410, a set of fitness scores (e.g., fitness scores 240 of FIG. 2) is generated for the set of services, wherein each respective fitness score from the set of fitness scores indicates a respective suitability of a respective service for the resident.

At block 1415, a machine learning model (e.g., machine learning model 140 of FIG. 1) is trained to generate residential service plans based at least in part on the set of fitness scores using one or more collaborative filtering techniques.

At block 1420, the trained machine learning model is deployed.

### Example Method for Generating Residential Service Plans Using Machine Learning

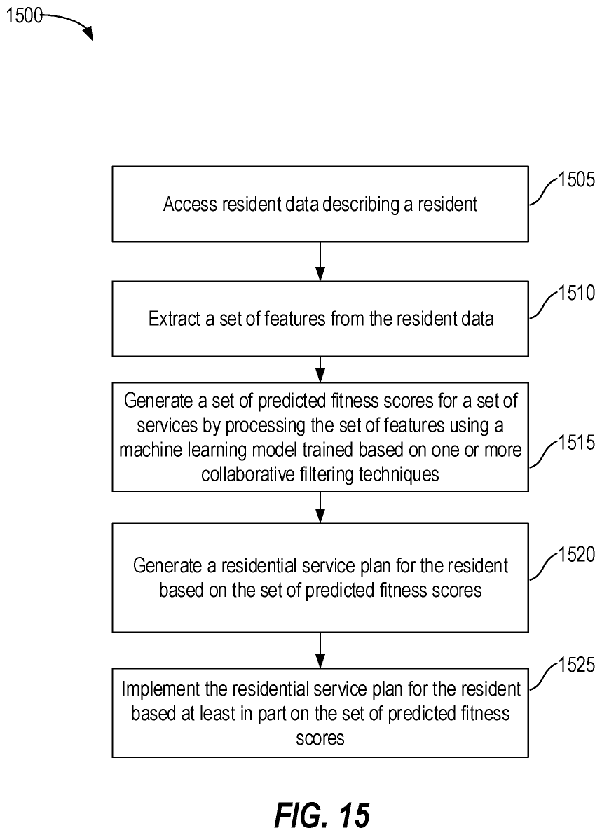

FIG. 15 is a flow diagram depicting an example method 1500 for generating residential service plans using machine learning models. In some embodiments, the method 1500 is performed by a machine learning system, such as the machine learning system 135 of FIG. 1, the machine learning system 335 of FIG. 3, and the like.

At block 1505, resident data (e.g., resident data 305 of FIG. 3) describing a resident is accessed.

At block 1510, a set of features is extracted from the resident data.

At block 1515, a set of predicted fitness scores is generated for a set of services by processing the set of features using a machine learning model trained based using one or more collaborative filtering techniques.

At block 1520, a residential service plan (e.g., service plan 340 of FIG. 3) is generated for the resident based on the set of predicted fitness scores.

At block 1525, the residential service plan is implemented for the resident based at least in part on the set of predicted fitness scores.

### Example Processing System for Improved Machine Learning

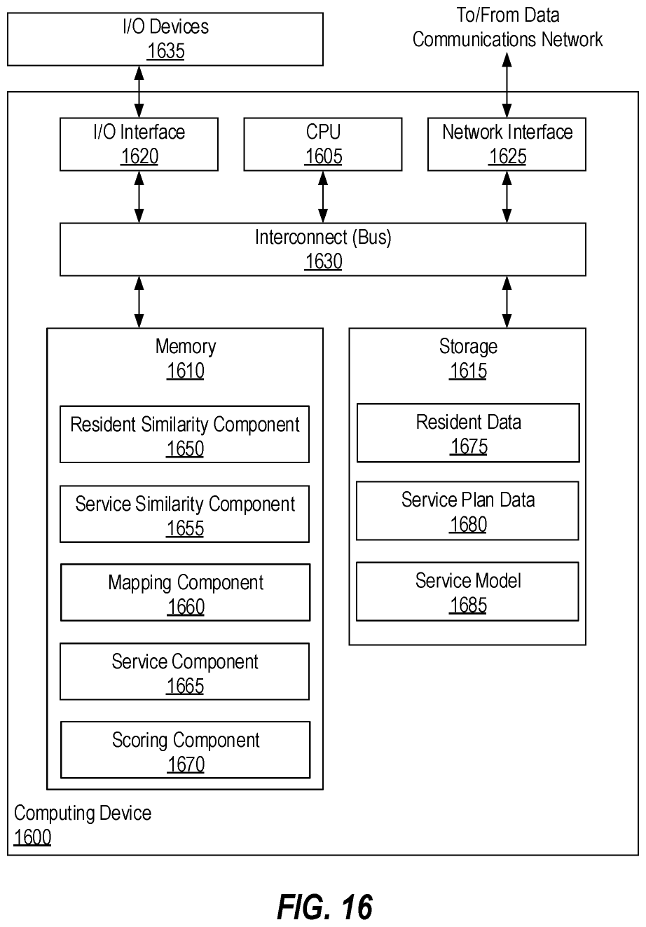

FIG. 16 depicts an example computing device 1600 configured to perform various aspects of the present disclosure. Although depicted as a physical device, in embodiments, the computing device 1600 may be implemented using virtual device(s), and/or across a number of devices (e.g., in a cloud environment). In one embodiment, the computing device 1600 corresponds to the machine learning system 135 of FIG. 1, and/or the machine learning system 335 of FIG. 3.

As illustrated, the computing device 1600 includes a CPU 1605, memory 1610, storage 1615, a network interface 1625, and one or more I/O interfaces 1620. In the illustrated embodiment, the CPU 1605 retrieves and executes programming instructions stored in memory 1610, as well as stores and retrieves application data residing in storage 1615. The CPU 1605 is generally representative of a single CPU and/or GPU, multiple CPUs and/or GPUs, a single CPU and/or GPU having multiple processing cores, and the like. The memory 1610 is generally included to be representative of a random access memory. Storage 1615 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1635 (such as keyboards, monitors, etc.) are connected via the I/O interface(s) 1620. Further, via the network interface 1625, the computing device 1600 can be communicatively coupled with one or more other devices and components (e.g., via a network, which may include the Internet, local network(s), and the like). As illustrated, the CPU 1605, memory 1610, storage 1615, network interface(s) 1625, and I/O interface(s) 1620 are communicatively coupled by one or more buses 1630.

In the illustrated embodiment, the memory 1610 includes a resident similarity component 1650 (e.g., the resident similarity component 410 of FIG. 4), a service similarity component 1655 (e.g., the service similarity component 425 of FIG. 4), a mapping component 1660 (e.g., the mapping component 435 of FIG. 4), a service component 1665 (e.g., the service component 510 of FIG. 5), and a scoring component 1670 (e.g., the sentiment component 220 of FIG. 2, scoring component 235 of FIG. 2, and/or scoring component 525 of FIG. 5), which may perform one or more embodiments discussed above. Although depicted as discrete components for conceptual clarity, in embodiments, the operations of the depicted components (and others not illustrated) may be combined or distributed across any number of components. Further, although depicted as software residing in memory 1610, in embodiments, the operations of the depicted components (and others not illustrated) may be implemented using hardware, software, or a combination of hardware and software.

For example, the resident similarity component 1650 and service similarity component 1655 may evaluate data to generate resident groups and service similarities, respectively, as discussed above with reference to FIGS. 4 and 9. The mapping component 1660 may generate mappings from resident groups to service sets, as discussed above with reference to FIGS. 4 and 9. The service component 1665 may evaluate resident attributes to generate a set of proposed or potential services, as discussed above. The scoring component 1670 may score historical services (e.g., to generate fitness scores used for training) and/or score proposed services (to generate predicted fitness scores for new service plans), as discussed above.

In the illustrated example, the storage 1615 includes resident data 1675 (which may correspond to historical data, such as historical data 105 of FIG. 1, resident data 305 of FIG. 3, resident attributes 405 of FIG. 4, and/or resident attributes 505 of FIG. 5), as well as service plan data 1680 (which may correspond to service plans 130 of FIG. 1, service data 205 of FIG. 2, service plans 340 of FIG. 3, resident services 420 of FIG. 4, and/or proposed services 515 of FIG. 5). The storage 1615 also includes a service model 1685 (which may correspond to the machine learning model 140 of FIG. 1). Although depicted as residing in storage 1615, the resident data 1675, service plan data 1680, and service model 1685 may be stored in any suitable location, including memory 1610.

### Example Clauses

Implementation examples are described in the following numbered clauses:

Clause 1: A method, comprising: accessing resident data describing a set of services received by a resident; generating a set of fitness scores for the set of services, wherein each respective fitness score from the set of fitness scores indicates a respective suitability of a respective service for the resident; training a machine learning model to generate residential service plans based at least in part on the set of fitness scores using one or more collaborative filtering techniques; and deploying the trained machine learning model.

Clause 2: The method of Clause 1, wherein generating the set of fitness scores comprises generating a first fitness score for a first service of the set of services, comprising: extracting one or more features, describing the first service, from the resident data; transforming at least one feature of the one or more features by applying one or more preprocessing operations; and generating the first fitness score based on the transformed at least one feature.

Clause 3: The method of any one of Clauses 1-2, wherein the one or more features comprise at least one of: (i) an amount of time spent providing the first service; (ii) one or more natural language notes relating to providing the first service; or (iii) a completion status of the first service.

Clause 4: The method of any one of Clauses 1-3, wherein transforming the at least one feature comprises generating a complexity score by processing the one or more natural language notes using one or more sentiment analysis models.

Clause 5: The method of any one of Clauses 1-4, wherein: the amount of time is directly related to the first fitness score, and the complexity score is directly related to the first fitness score.

Clause 6: The method of any one of Clauses 1-5, further comprising: extracting, from the resident data, a plurality of resident attributes describing the resident; and training the machine learning model based further on the plurality of resident attributes.

Clause 7: The method of any one of Clauses 1-6, wherein the machine learning model is further trained based on data for a plurality of residents, comprising: extracting a respective plurality of resident attributes for each respective resident of the plurality of residents; generating a set of resident groups based on the respective pluralities of resident attributes; generating a plurality of service groups, from the set of services, based on co-occurrences of services in the data for the plurality of residents; and mapping each respective resident group of the set of resident groups to a corresponding subset of services based at least in part on the plurality of service groups.

Clause 8: A method, comprising: accessing resident data describing a resident; generating a residential service plan for the resident, comprising: extracting a set of features from the resident data; and generating a set of predicted fitness scores for a set of services by processing the set of features using a machine learning model trained based on one or more collaborative filtering techniques; and implementing the residential service plan for the resident based at least in part on the set of predicted fitness scores.

Clause 9: The method of Clause 8, wherein generating the set of predicted fitness scores for the set of services comprises: assigning the resident to a first resident group, of a plurality of resident groups indicated in the machine learning model, based on the set of features; identifying the set of services based on determining that the set of services is associated with the first resident group in the machine learning model; and generating the set of predicted fitness scores based on historical fitness scores used to train the machine learning model.

Clause 10: The method of any one of Clauses 8-9, wherein implementing the residential service plan comprises: outputting the set of predicted fitness scores to a care provider; receiving selection, from the care provider, of a subset of services from the set of services; and scheduling the subset of services for the resident.

Clause 11: The method of any one of Clauses 8-10, wherein outputting the set of predicted fitness scores comprises displaying the set of services and the set of predicted fitness scores on a graphical user interface (GUI), comprising, for each respective service of the set of services: selecting a manner of presentation based on a corresponding predicted fitness score from the set of predicted fitness scores; and generating a visual depiction of suitability of the respective service for the resident based on the selected manner of presentation.

Clause 12: The method of any one of Clauses 8-11, further comprising: generating a plurality of residential service plans for a plurality of residents in a residential care facility; generating an aggregate service plan based on the plurality of residential service plans; and facilitating staff allocation based on the aggregate service plan.

Clause 13: The method of any one of Clauses 8-12, subsequent to implementing the residential service plan, generating a first fitness score for a first service of the set of services, comprising: extracting one or more features, describing the first service, from resident data for the resident; transforming at least one feature of the one or more features by applying one or more preprocessing operations; and generating the first fitness score based on the transformed at least one feature.

Clause 14: The method of any one of Clauses 8-13, wherein the one or more features comprise at least one of: (i) an amount of time spent providing the first service; (ii) one or more natural language notes relating to providing the first service; or (iii) a completion status of the first service.

Clause 15: The method of any one of Clauses 8-14, wherein transforming the at least one feature comprises generating a complexity score by processing the one or more natural language notes using one or more sentiment analysis models.

Clause 16: The method of any one of Clauses 8-15, wherein: the amount of time is directly related to the first fitness score, and the complexity score is directly related to the first fitness score.

Clause 17: The method of any one of Clauses 8-16, further comprising refining the machine learning model based on the first fitness score.

Clause 18: A system, comprising: one or more computer processors; and one or more memories containing a program which when executed by the one or more computer processors performs an operation, the operation comprising: accessing resident data describing a resident; generating a residential service plan for the resident, comprising: extracting a set of features from the resident data; and generating a set of predicted fitness scores for a set of services by processing the set of features using a machine learning model trained based on one or more collaborative filtering techniques; and implementing the residential service plan for the resident based at least in part on the set of predicted fitness scores, comprising, for each respective service of the set of services: selecting a manner of presentation based on a corresponding predicted fitness score from the set of predicted fitness scores; and generating a visual depiction, on a graphical user interface (GUI), of suitability of the respective service for the resident based on the selected manner of presentation.

Clause 19: The system of Clause 18, wherein generating the set of predicted fitness scores for the set of services comprises: assigning the resident to a first resident group, of a plurality of resident groups indicated in the machine learning model, based on the set of features; identifying the set of services based on determining that the set of services is associated with the first resident group in the machine learning model; and generating the set of predicted fitness scores based on historical fitness scores used to train the machine learning model.

Clause 20: The system of any one of Clauses 18-19, wherein implementing the residential service plan further comprises: outputting the set of predicted fitness scores to a care provider, comprising displaying the set of services and the set of predicted fitness scores on the GUI; receiving selection, from the care provider, of a subset of services from the set of services; and scheduling the subset of services for the resident.

Clause 21: A system, comprising: a memory comprising computer-executable instructions; and one or more processors configured to execute the computer-executable instructions and cause the processing system to perform a method in accordance with any one of Clauses 1-20.

Clause 22: A system, comprising means for performing a method in accordance with any one of Clauses 1-20.

Clause 23: A non-transitory computer-readable medium comprising computer-executable instructions that, when executed by one or more processors of a processing system, cause the processing system to perform a method in accordance with any one of Clauses 1-20.

Clause 24: A computer program product embodied on a computer-readable storage medium comprising code for performing a method in accordance with any one of Clauses 1-20.

### Additional Considerations

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or systems (e.g., the machine learning system 135) or related data available in the cloud. For example, the machine learning system could execute on a computing system in the cloud and train and/or use machine learning models. In such a case, the machine learning system 135 could train models to generate and evaluate service plans, and store the models at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
accessing resident data describing a set of services received by a resident;
generating a set of fitness scores for the set of services, wherein each respective fitness score from the set of fitness scores indicates a respective suitability of a respective service for the resident;
training a machine learning model using one or more collaborative filtering techniques to generate residential service plans based at least in part on the set of fitness scores; and
deploying the trained machine learning model.

2. The method of claim 1, wherein generating the set of fitness scores comprises generating a first fitness score for a first service of the set of services, comprising:
extracting one or more features, describing the first service, from the resident data;
transforming at least one feature of the one or more features by applying one or more preprocessing operations; and
generating the first fitness score based on the transformed at least one feature.

3. The method of claim 2, wherein the one or more features comprise at least one of:
(i) an amount of time spent providing the first service;
(ii) one or more natural language notes relating to providing the first service; or
(iii) a completion status of the first service.

4. The method of claim 3, wherein transforming the at least one feature comprises generating a complexity score by processing the one or more natural language notes using one or more sentiment analysis models.

5. The method of claim 4, wherein:
the amount of time is directly related to the first fitness score, and
the complexity score is directly related to the first fitness score.

6. The method of claim 1, further comprising:
extracting, from the resident data, a plurality of resident attributes describing the resident; and
training the machine learning model based further on the plurality of resident attributes.

7. The method of claim 6, wherein the machine learning model is further trained based on data for a plurality of residents, comprising:
extracting a respective plurality of resident attributes for each respective resident of the plurality of residents;
generating a set of resident groups based on the respective pluralities of resident attributes;
generating a plurality of service groups, from the set of services, based on co-occurrences of services in the data for the plurality of residents; and
mapping each respective resident group of the set of resident groups to a corresponding subset of services based at least in part on the plurality of service groups.

8. A method, comprising:
accessing resident data describing a resident;
generating a residential service plan for the resident, comprising:
extracting a set of features from the resident data; and
generating a set of predicted fitness scores for a set of services by processing the set of features using a machine learning model trained based on one or more collaborative filtering techniques; and
implementing the residential service plan for the resident based at least in part on the set of predicted fitness scores.

9. The method of claim 8, wherein generating the set of predicted fitness scores for the set of services comprises:
assigning the resident to a first resident group, of a plurality of resident groups indicated in the machine learning model, based on the set of features;
identifying the set of services based on determining that the set of services is associated with the first resident group in the machine learning model; and
generating the set of predicted fitness scores based on historical fitness scores used to train the machine learning model.

10. The method of claim 8, wherein implementing the residential service plan comprises:
outputting the set of predicted fitness scores to a care provider;
receiving selection, from the care provider, of a subset of services from the set of services; and
scheduling the subset of services for the resident.

11. The method of claim 10, wherein outputting the set of predicted fitness scores comprises displaying the set of services and the set of predicted fitness scores on a graphical user interface (GUI), comprising, for each respective service of the set of services:
selecting a manner of presentation based on a corresponding predicted fitness score from the set of predicted fitness scores; and
generating a visual depiction of suitability of the respective service for the resident based on the selected manner of presentation.

12. The method of claim 8, further comprising:
generating a plurality of residential service plans for a plurality of residents in a residential care facility;
generating an aggregate service plan based on the plurality of residential service plans; and
facilitating staff allocation based on the aggregate service plan.

13. The method of claim 8, further comprising, subsequent to implementing the residential service plan, generating a first fitness score for a first service of the set of services, comprising:
extracting one or more features, describing the first service, from resident data for the resident;
transforming at least one feature of the one or more features by applying one or more preprocessing operations; and
generating the first fitness score based on the transformed at least one feature.

14. The method of claim 13, wherein the one or more features comprise at least one of:
(i) an amount of time spent providing the first service;
(ii) one or more natural language notes relating to providing the first service; or
(iii) a completion status of the first service.

15. The method of claim 14, wherein transforming the at least one feature comprises generating a complexity score by processing the one or more natural language notes using one or more sentiment analysis models.

16. The method of claim 15, wherein:
the amount of time is directly related to the first fitness score, and
the complexity score is directly related to the first fitness score.

17. The method of claim 13, further comprising refining the machine learning model based on the first fitness score.

18. A system, comprising:
one or more computer processors; and
one or more memories containing a program which when executed by the one or more computer processors performs an operation, the operation comprising:
accessing resident data describing a resident;
generating a residential service plan for the resident, comprising:
extracting a set of features from the resident data; and
generating a set of predicted fitness scores for a set of services by processing the set of features using a machine learning model trained based on one or more collaborative filtering techniques; and
implementing the residential service plan for the resident based at least in part on the set of predicted fitness scores, comprising, for each respective service of the set of services:
selecting a manner of presentation based on a corresponding predicted fitness score from the set of predicted fitness scores; and
generating a visual depiction, on a graphical user interface (GUI), of suitability of the respective service for the resident based on the selected manner of presentation.

19. The system of claim 18, wherein generating the set of predicted fitness scores for the set of services comprises:
assigning the resident to a first resident group, of a plurality of resident groups indicated in the machine learning model, based on the set of features;
identifying the set of services based on determining that the set of services is associated with the first resident group in the machine learning model; and
generating the set of predicted fitness scores based on historical fitness scores used to train the machine learning model.

20. The system of claim 18, wherein implementing the residential service plan further comprises:
outputting the set of predicted fitness scores to a care provider, comprising displaying the set of services and the set of predicted fitness scores on the GUI;
receiving selection, from the care provider, of a subset of services from the set of services; and
scheduling the subset of services for the resident.
